(19) United States
(12) Patent Application Publication
(10) Pub. No.: US 2026/0290544 A1
(43) Pub. Date: Sep. 24, 2026

(54) SYSTEM

(71) Applicant: SoftBank Group Corp., Tokyo (JP)

(72) Inventor: Hiroe SHIMANE, Tokyo (JP)

(73) Assignee: SoftBank Group Corp., Tokyo (JP)

(21) Appl. No.: 19/564,237

(22) Filed: Mar. 12, 2026

(30) Foreign Application Priority Data
Mar. 19, 2025 (JP) 2025-044892

(51) Int. Cl. G16H 20/60; G16H 10/60

(52) U.S. Cl. CPC G16H 20/60; G16H 10/60

(57) ABSTRACT

A system includes a processor that is configured to receive intake information of a plurality of nutritional supplements and evaluate, by using a generative AI model, potential health risks when the nutritional supplements are taken over a long term, issue a warning to a user based on a result of the evaluation, and receive a health examination result from the user and propose, by using a generative AI model based on the health examination result, nutritional supplements that are not recommended for intake and nutritional supplements that are recommended for intake.

### CROSS-REFERENCE TO RELATED APPLICATION

This application is based on and claims priority under 35 USC 119 from Japanese Patent Application No. 2025-044892 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional systems for managing nutritional supplements mainly rely on static, rule-based assessments or simple threshold checks of individual ingredients. Such systems do not adequately account for complex, personalized risk factors, such as long-term combined intake of multiple supplements, dynamic changes in a user's health status as reflected in health examination results, or time-varying biometric and emotional states. As a result, users may unknowingly continue supplement regimens that pose potential long-term health risks, may not receive appropriate guidance regarding which supplements should be avoided or recommended in view of their current medical condition, and may lack objective feedback on the actual effectiveness of the supplements they are taking.

In particular, there is a need for a system that: (i) evaluates long-term potential health risks arising from a plurality of nutritional supplements taken in combination, (ii) flexibly proposes supplements to avoid and supplements to take based on individualized health examination data, and (iii) quantitatively and qualitatively evaluates supplement effects by correlating intake information with continuously acquired biometric information and emotional state information. Existing technologies do not sufficiently leverage generative AI models to holistically infer and explain such risks and effects in a personalized, data-driven manner. Therefore, an improved technique is required that uses generative AI models to perform integrated analysis of supplement intake information, health examination results, biometric information, and emotional state information, and to provide the user with adaptive warnings and recommendations.

### SUMMARY

To solve the above-described problems, the present invention provides a system comprising a processor, wherein the processor is configured to receive intake information of a plurality of nutritional supplements and evaluate, by using a generative AI model, potential health risks when the nutritional supplements are taken over a long term. The processor is further configured to issue a warning to a user based on a result of the evaluation, such that the user is notified when a combination, dosage, or duration of nutritional supplement intake may increase a long-term health risk.

In addition, the processor is configured to receive a health examination result from the user and propose, by using the generative AI model based on the health examination result, nutritional supplements that are not recommended for intake and nutritional supplements that are recommended for intake. By incorporating blood test values, physical measurements, and other clinical parameters into the inputs of the generative AI model, the system generates individualized recommendations that reflect the user's current health status and medical risks.

Furthermore, in one aspect, the processor is configured to continuously measure biometric information of the user, such as blood pressure and heart rate, and to evaluate, by using the generative AI model, an effect of the nutritional supplements by correlating the biometric information with the intake information of the nutritional supplements. By analyzing temporal trends of biometric data before and after initiation or modification of supplement intake, the system estimates the effectiveness and potential adverse influence of the supplements on physiological parameters.

In another aspect, the processor is configured to analyze an emotional state of the user, and to evaluate, by using the generative AI model, an effect of the nutritional supplements by combining the emotional state with the intake information of the nutritional supplements. For example, the emotional state may be inferred from user-input questionnaires, voice or text content, or sensor-derived indicators. By correlating changes in emotional state with supplement intake patterns, the system can infer potential effects of supplements on mood, stress, or mental well-being.

Through these means, the system according to the present invention uses a generative AI model as a core inference engine to integrate multiple heterogeneous data sources, including supplement intake information, health examination results, biometric information, and emotional state information, to provide comprehensive, personalized risk evaluations, warnings, and recommendations regarding nutritional supplements.

The term “system” refers to an integrated combination of hardware and software components, including at least one processor and associated memory, communication interfaces, and storage, configured to execute the processing described in the present specification.

The term “processor” refers to any hardware logic component or combination of components capable of executing instructions, including but not limited to a central processing unit (CPU), a graphics processing unit (GPU), a digital signal processor (DSP), an application-specific integrated circuit (ASIC), a field-programmable gate array (FPGA), or a plurality of such components operating together.

The term “user” refers to a human individual who takes or plans to take nutritional supplements and who interacts with the system to provide information, receive warnings, and obtain recommendations.

The term “nutritional supplement” refers to any ingestible product, including but not limited to vitamins, minerals, amino acids, herbal products, functional foods, and other dietary or health-support products, that is intended to supplement the diet and is not generally classified as a prescription pharmaceutical drug.

The term “intake information” refers to information indicating at least one aspect of how a user takes a nutritional supplement, including, for example, the type or name of the supplement, the ingredients contained therein, the dosage amount, the intake frequency, the intake schedule, and the start date and/or duration of intake.

The term “health examination result” refers to information obtained from a medical or health checkup, including but not limited to blood test values, urine test values, imaging results, physical measurements such as height, weight, body mass index, blood pressure, and any other clinically relevant parameters that characterize the health status of the user at a certain time.

The term “biometric information” refers to physiological data measured from the body of the user, including but not limited to blood pressure, heart rate, heart rate variability, respiratory rate, body temperature, activity level, sleep state, and other vital signs, which may be acquired continuously or intermittently by sensors or wearable devices.

The term “emotional state” refers to a condition representing the psychological or affective status of the user, including but not limited to mood, stress level, anxiety level, depression level, or emotional valence, which may be inferred from questionnaires, behavioral patterns, biometric indicators, voice or text analysis, or other observable signals.

The term “generative AI model” refers to a machine learning model, such as a neural network-based model, that is trained on data to generate outputs including evaluations, predictions, recommendations, explanatory text, or other synthesized information, in response to input data including intake information, health examination results, biometric information, and emotional state information.

The term “potential health risk” refers to a likelihood or possibility of an adverse effect on the health of the user that may arise from the intake of nutritional supplements, particularly when such supplements are taken at certain dosages, in certain combinations, or over a long-term period, and includes risks related to toxicity, organ burden, interactions, or exacerbation of existing medical conditions.

The term “long term” refers to a period of nutritional supplement intake that extends for a duration sufficient to cause cumulative physiological effects or to raise concerns regarding chronic exposure, such duration being defined in the system according to preset thresholds or learned criteria and typically spanning weeks, months, or longer.

The term “warning” refers to any notification, alert, or message generated by the system and presented to the user to indicate the presence or possibility of a potential health risk, an undesirable interaction, an excessive intake, or any other condition for which the user's attention or action is advised.

The term “supplements that are not recommended for intake” refers to nutritional supplements that the system identifies, based on the health examination result, intake information, biometric information, emotional state, or learned patterns of the generative AI model, as being undesirable or risky for the user to take or continue taking.

The term “supplements that are recommended for intake” refers to nutritional supplements that the system identifies, based on the health examination result, intake information, biometric information, emotional state, or learned patterns of the generative AI model, as being desirable or beneficial for the user to take or consider taking, subject to any applicable medical or regulatory constraints.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

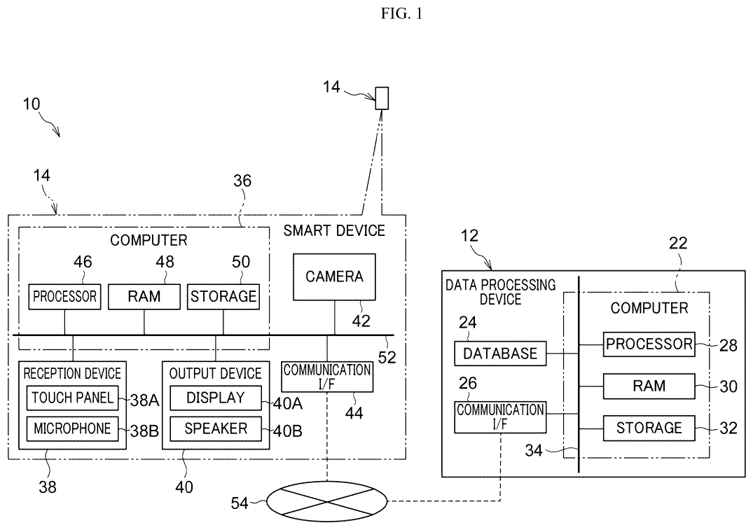

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

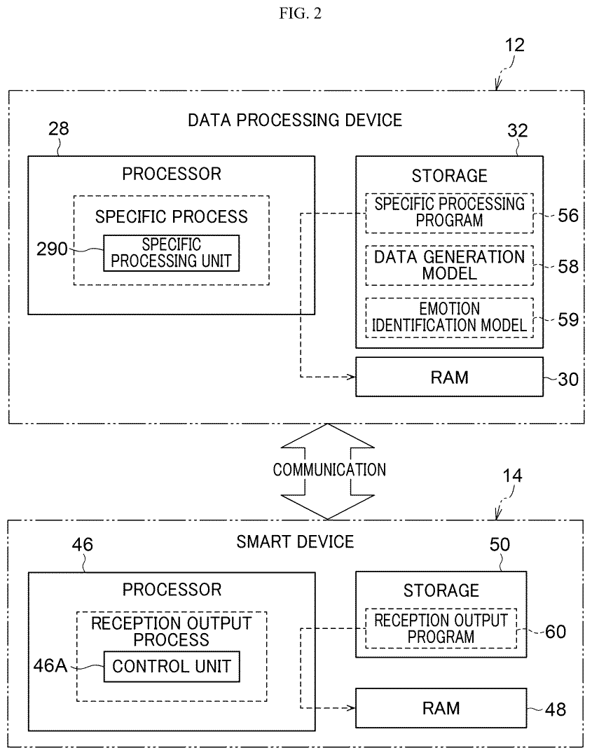

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented systems for managing intake of dietary supplements typically rely on static rule sets and simplistic threshold checks executed by generic application logic. Such systems often perform only basic comparisons between reported intake amounts and fixed reference values, and then return canned warning messages. As a result, these systems suffer from several technical limitations in terms of data processing efficiency, adaptability, and quality of machine-generated outputs.

First, conventional systems do not systematically normalize and structure heterogeneous intake information originating from user terminals. Users may input free-form text, inconsistent units, and irregular frequency expressions. When this information is processed directly by the server without robust normalization and structuring, the server must perform repetitive and ad hoc parsing and validation operations. This leads to increased processing overhead, higher error rates in data interpretation, and difficulty in reliably aggregating intake amounts across time periods or across multiple components.

Second, existing architectures typically treat risk calculation and explanatory message generation as either fully rule-based or fully delegated to a generic machine learning service, without an integrated control mechanism. When generic generative models are simply exposed to raw or semi-structured intake data, the models may produce verbose or inconsistent warnings that are disconnected from precise numerical risk evaluations.

Conversely, when systems rely solely on template-based messages, they are unable to adapt the content to complex combinations of components, long-term intake patterns, or nuanced levels of risk. This mismatch between numeric evaluation and text generation results in low-quality feedback to the user and inefficient utilization of computing resources.

Third, conventional systems do not provide a tightly coupled feedback loop between the user terminal and the server that is optimized for iterative risk reevaluation. User corrections to intake information are often treated as independent submissions, which forces the server to redundantly process large amounts of repeated data and to regenerate explanations from scratch. In addition, there is no technical mechanism for dynamically choosing between lightweight template-based warning generation and heavier generative AI-based explanation generation, based on measured risk severity. This lack of adaptive processing causes unnecessary computational load, increased latency, and suboptimal use of specialized hardware or external inference services.

Fourth, known techniques for integrating generative AI models often do not define a structured, machine-oriented way of constructing prompt sentences from numerical risk data. Unstructured prompts may omit important contextual information, include personally identifiable information, or fail to constrain the model to produce concise and relevant explanations. This leads to unstable output, difficulty in post-processing the generated text, and challenges in meeting compliance or safety policies within the system.

Accordingly, there is a need for an improved computer-implemented system and server-side architecture that (i) converts user-provided supplement intake information from terminals into normalized structured data, (ii) performs efficient and reliable aggregation and risk computation against stored medical standard information, (iii) systematically constructs prompt sentences for a generative AI model based on computed risk metrics, (iv) selectively uses either template-based or generative-AI-based warning generation according to risk severity, and (v) supports an efficient iterative feedback loop where updated user inputs trigger targeted recomputation and updated warnings. By addressing these technical issues, the system can improve the overall accuracy, performance, and robustness of health risk evaluation and warning generation in a computer network environment.

The specific processing by the specific processing unit 290 of the data processing device 12 in Example 1 is realized by the following means.

The present invention provides a server comprising a processor configured to receive structured intake information for dietary supplements from a user terminal via a communication interface; to perform format validation and content normalization on the structured intake information so as to convert heterogeneous user inputs into normalized records keyed by user identifiers; to store the normalized intake records and medical standard information, including intake upper limits, recommended intake amounts, and component interaction rules, in at least one storage device; to aggregate, for each component, intake amounts over predetermined time periods by executing arithmetic operations on the normalized records retrieved from the storage device; to compare the aggregated intake amounts with the medical standard information in order to compute, by rule-based logic, a numerical risk level for each component and an interaction risk between multiple components; to generate, based on the computed numerical results, an evaluation input sentence in which the numerical risk data and component identifiers are encoded as a prompt sentence suitable for input to a generative AI model; to selectively execute, according to a severity level of the computed risk, either (i) a template-based warning generation process that formats warning information directly from the numerical risk data, or (ii) a generative-AI-based explanation generation process that transmits the prompt sentence to the generative AI model and receives, from the generative AI model, a response sentence including a natural language explanation and recommended actions; to merge, in the case of the generative-AI-based explanation generation process, the response sentence with the numerical risk data into warning information including component identifiers, numerical details, danger categories, and warning content; and to transmit the generated warning information to the user terminal for display. This enables improved computer operation in that the server-side processing pipeline normalizes and structures incoming data for efficient aggregation, dynamically selects between computationally lightweight template-based processing and more resource-intensive generative model inference, and systematically constructs and constrains prompt sentences to obtain stable, contextually aligned natural language output from the generative AI model, thereby reducing processing overhead, improving accuracy and consistency of risk evaluation and explanations, and supporting a responsive iterative feedback loop between the user terminal and the server.

The term “system” refers to a combination of hardware resources, software resources, and communication resources that cooperatively execute processing for intake management, risk evaluation, and warning generation regarding dietary supplements.

The term “processor” refers to a hardware execution unit, such as a central processing unit or other programmable computation device, that executes instructions of a program to perform data processing operations defined in this specification.

The term “user terminal” refers to an information processing device operated by a user, such as a mobile device, a portable information processing device, or a desktop information processing device, that provides a user interface for inputting intake information and displaying warning information.

The term “server” refers to an information processing apparatus, typically located remotely from the user terminal and connected via a network, that performs centralized storage, aggregation, risk evaluation, and warning generation based on intake information received from multiple user terminals.

The term “communication path” refers to a logical or physical communication channel, including wired or wireless network infrastructure, that enables data exchange between the user terminal and the server using a communication protocol.

The term “intake information” refers to information representing the manner in which a user ingests dietary supplements, including at least one of a product identifier, a component identifier, an intake amount, an intake unit, and an intake frequency.

The term “structured information” refers to intake information that has been formatted in accordance with a predefined data schema, such as a structured data object or a serialized document, in which individual items of intake information are represented as fields with explicit attribute names and data types.

The term “format validation” refers to processing that checks whether received structured information conforms to a predetermined syntax or format, including verification of required fields, data types, and allowable value ranges.

The term “content normalization” refers to processing that converts heterogeneous or inconsistent representations of intake information into standardized internal representations, including at least one of unit conversion, normalization of naming conventions, and canonicalization of component identifiers.

The term “record information” refers to normalized intake information and related metadata that are stored in a storage device in association with user identifiers and timestamps, and that can be retrieved for subsequent aggregation and analysis.

The term “medical standard information” refers to reference information that defines at least one of an upper intake limit, a recommended intake amount, and an interaction rule for each component, and that is used as a basis for computing risk levels in the system.

The term “component” refers to an identifiable constituent substance of a dietary supplement, such as a nutrient, a vitamin, a mineral, or a functional ingredient, for which an intake amount and a health-related standard are defined.

The term “component identifier” refers to data that uniquely or canonically identifies a component within the system, such as a standardized name, a code, or another normalized label used for aggregation and comparison.

The term “aggregation” refers to processing that combines multiple instances of intake information for a given component over a predetermined time period to derive cumulative or average amounts by applying arithmetic operations.

The term “predetermined period” refers to a time interval defined by system configuration or program logic, such as a day, a week, or a month, over which component intake amounts are aggregated.

The term “risk level” refers to a categorical or numerical indicator representing the degree of potential health risk associated with a component's intake, calculated by comparing aggregated intake amounts with medical standard information.

The term “interaction risk” refers to a degree of potential health risk associated with a combination of two or more components, determined based on interaction rules in the medical standard information and the user's combined intake of the components.

The term “evaluation input sentence” refers to a text representation that encodes numerical risk data, component identifiers, and contextual information in a natural language form suitable as input to a generative AI model.

The term “prompt sentence” refers to a type of evaluation input sentence that is specifically structured and formatted to instruct a generative AI model to generate an output, such as an explanation of health risks and recommended actions.

The term “generative AI model” refers to a trained machine learning model that receives text-based input and generates text-based output using a generative algorithm, such as a probabilistic language model implemented by a neural network.

The term “response sentence” refers to a natural language text generated by the generative AI model in response to a prompt sentence, and that may include at least one of an explanation of health risks and a recommendation for user behavior.

The term “warning information” refers to structured data generated by the server that includes at least one of a component identifier, numerical risk details, a danger category, and warning content, and that is transmitted to the user terminal for presentation.

The term “danger category” refers to a classification label assigned to a component or a set of components based on a computed risk level and interaction risk, indicating a degree of caution, such as a safe level, a caution level, or a high-risk level.

The term “warning content” refers to textual data describing a risk, a reason for the risk, and at least one recommended action or caution to be presented to a user.

The term “template-based warning generation process” refers to processing in which warning information is generated from numerical risk data by inserting the data into predefined text patterns, without using a generative AI model for text generation.

The term “explanation generation process” refers to processing that uses a generative AI model to generate a natural language explanation or recommendation based on a prompt sentence constructed from numerical risk data and component identifiers.

The term “severity” refers to a measure of seriousness of a computed risk level or interaction risk, which is used by the system to determine whether to use template-based warning generation or generative-AI-based explanation generation.

The term “display screen” refers to a visual output interface of the user terminal that can present warning information, including danger categories and explanations, in a human-perceivable form.

The term “visual distinction” refers to a graphical or layout-based differentiation, such as color coding, highlighting, or icon usage, that indicates different danger categories when presenting warning information on the display screen.

The term “iterative feedback loop” refers to a sequence of operations in which warning information is presented to a user, the user modifies intake information in response, and the server re-executes risk evaluation and warning generation based on the modified intake information.

In one embodiment, the system includes at least one server, a plurality of terminals, and a communication network connecting the server and the terminals. The server includes a processor, a main memory, a non-volatile storage device, and a network interface. The terminal includes a processor, a memory, a display device, an input device, and a wireless or wired communication interface. The terminal executes an application program for supplement management, and the server executes a server-side program for intake aggregation, risk computation, prompt sentence generation, and interaction with a generative AI model.

The terminal uses a mobile operating system or a general-purpose operating system and executes an application implemented, for example, as a mobile application or a web application. The terminal presents input user interfaces implemented with a user interface framework, such as a mobile UI toolkit or a web UI library. The terminal allows the user to enter intake information including at least a supplement name, a component name, an intake amount, a unit (for example, milligrams, international units), and an intake frequency (for example, times per day, times per week). The terminal converts the raw user input into a structured internal data model, such as an object or a record, and then serializes this model into structured information, for example a JSON-like logical data structure, using a serialization library provided by the operating environment.

The terminal performs local validation of the intake information by checking that numeric fields contain valid numeric values within plausible ranges and that mandatory fields are not empty. The terminal normalizes some user interface expressions into canonical representations, for example by mapping “once a day” to a numeric frequency value 1.0 and by mapping variations of unit expressions to standardized unit codes. The terminal transmits the structured and partially normalized information to the server through the network interface using an HTTP-based or similar application-layer protocol over a secure channel such as Transport Layer Security.

The server receives the structured intake information through a network interface and passes it to a server-side application framework. The server parses the serialized data using a parser library and converts it into internal data structures such as records, arrays, or objects held in main memory. The server executes format validation by checking presence of required fields (user identifier, component identifier, intake amount, unit, frequency) and by verifying that the types of the fields match expected types. The server executes content normalization by applying a normalization module that converts heterogeneous representations into canonical forms. For example, the server converts all intake amounts for a given component into a common base unit by multiplying or dividing by predetermined conversion factors, and the server maps component names entered by the user to canonical component identifiers stored in a reference table using string normalization and lookup operations.

The server stores the normalized intake records in a storage device such as a relational database or a key-value store. The server associates each record with a user identifier and a timestamp, and stores it in tables or collections optimized for subsequent aggregation. The server also stores medical standard information, which may include upper intake limits, recommended intake amounts, and component interaction rules, in dedicated tables or data structures. The medical standard information may be loaded into a main-memory cache to reduce access latency. By normalizing the intake records at write time, the server reduces the amount of repeated conversion and parsing required for each subsequent risk evaluation, thereby improving processing speed and reducing error probability.

The server aggregates intake amounts by executing arithmetic data processing on the normalized records. The server retrieves, for a particular user and time range, all intake records from the storage device using indexed queries. The server groups the records by component identifier and computes cumulative and average intake amounts over predetermined periods such as daily, weekly, and monthly intervals. The server performs numeric operations such as summation and division to derive these aggregate values. Because the records are already normalized to canonical units and component identifiers, the aggregation can be performed as straightforward numeric computation without repeated unit conversion or text matching. This yields a technical effect of reducing CPU cycles and memory accesses per evaluation and enabling efficient evaluation for many users in parallel.

The server compares the aggregated intake amounts with the medical standard information stored in the storage device. The server uses rule-based logic implemented as a set of conditional evaluations. For each component, the server obtains from the medical standard information an upper intake limit and a recommended intake amount. The server computes, for each time period, whether the aggregated intake exceeds the upper limit and by how much.

The server also examines predefined component interaction rules, which specify pairs or sets of components and associated interaction conditions, such as that simultaneous long-term intake above certain thresholds increases risk. The server then computes a risk level for each component and an interaction risk level for each relevant combination based on these comparisons, and assigns danger categories such as “no risk,” “low,” “moderate,” or “high.”

The risk computation uses arithmetic comparisons and logical conjunction, disjunction, and thresholding, and produces structured numerical output that is machine-readable.

The server generates an evaluation input sentence that encodes the numeric risk data and context in natural language form for use as a prompt sentence to the generative AI model. The server constructs the prompt sentence by concatenating textual templates with filled-in variables representing component identifiers, intake amounts, upper limits, and danger categories. The server ensures that user identifiers or other personally identifiable information are omitted or anonymized. This structured prompt generation differs from arbitrary free-form prompting, because the server enforces a stable, schema-driven mapping from numeric data fields to sentence segments. This mapping improves reproducibility of outputs and allows the generative AI model to focus on interpreting the numeric context instead of guessing missing structure.

The server, in one embodiment, uses a generative AI model implemented as a transformer-based neural network. The server stores, in a model storage, model parameters including weight matrices and bias vectors for multiple attention layers and feed-forward layers. The generative AI model accepts tokenized text as input and outputs a sequence of tokens representing generated text. The server executes the model on specialized hardware such as a graphics processing unit or a neural processing unit to accelerate matrix multiplications and attention computations.

The server constructs token sequences by encoding the prompt sentence with a tokenizer that maps subword units to integer token identifiers. The generative AI model then processes the sequence through an embedding layer, a stack of transformer blocks with multi-head self-attention, layer normalization, and feed-forward sublayers, and an output projection layer.

During inference, the model applies a next-token prediction objective, where the probability distribution of the next token is computed using the softmax function over the output log its.

The server selects tokens based on a sampling method such as greedy decoding or top-k sampling with temperature control. The server can set decoding parameters such as maximum output length and temperature to ensure that generated explanations are concise and stable.

The generative AI model is trained before deployment on a training dataset including domain-specific text related to nutrition, medical guidelines, and explanatory language. The training process uses supervised learning where the model minimizes a cross-entropy loss function between predicted token distributions and target tokens. The model parameters are updated using gradient-based optimization such as stochastic gradient descent with adaptive learning rate. The training dataset may be augmented by synthetic examples where structured numeric data are paired with human-written explanations, thereby enabling the model to better interpret aggregated intake amounts and medical standard information. The training details are fixed prior to deployment, and at runtime the server executes only inference, not training. This clear separation enables predictable runtime performance and compliance with resource constraints.

The server, based on configuration and risk severity, selects between a template-based warning generation process and a generative-AI-based explanation generation process. For low or no risk levels, the server may generate warnings or confirmations using simple templates, without calling the generative AI model, thereby reducing computational load and latency. For higher risk levels or for complex interaction risks, the server transmits the prompt sentence to the generative AI model and obtains a response sentence that includes an explanation of potential long-term health risks and recommended actions. This selective use of the generative AI model improves overall system throughput because the server avoids calling the model for trivial cases where template-based messages are sufficient.

The server merges the response sentence from the generative AI model with the numerical risk data into warning information. The warning information includes, for each relevant component or component combination, a component identifier, numeric details such as aggregated intake versus upper limit, a danger category, and warning content. The warning content may incorporate or be based on the response sentence. The server encapsulates this warning information into a structured response and transmits it to the terminal via the network interface. Because the warning information includes both machine-readable numeric fields and human-readable explanatory text, subsequent processing such as logging, trend analysis, and UI rendering can be efficiently implemented.

The terminal receives the warning information from the server and parses it into internal data structures. The terminal displays the warning information on the display device. The terminal uses visual distinction, such as color coding or icons, to indicate different danger categories.

The terminal presents the explanation generated based on the prompt sentence and the generative AI model output in a text area that the user can scroll. The terminal also presents interactive controls that enable the user to modify the intake information, such as changing the intake amount, adjusting the frequency, or removing a supplement. The terminal then transmits modified intake information back to the server.

The user views the warning information and interacts with the terminal to adjust supplement intake. The user can iteratively modify the intake information and immediately receive updated risk evaluations and warnings. This iterative feedback loop, in which the server reuses prior normalized records and performs focused recomputation for only the modified data, reduces redundant processing compared to conventional systems that reprocess entire histories each time. The system therefore achieves technical effects including improved response time for the user, reduced server load for large user populations, and more accurate risk assessments due to normalized and aggregated data.

As a concrete example, the user inputs, through the terminal, that the user takes “Vitamin A 10000 IU daily, Vitamin C 1000 mg daily, Vitamin D 2000 IU daily, and Zinc 50 mg daily.”

The terminal converts this to structured information and sends it to the server. The server normalizes units, associates each intake with canonical component identifiers, stores the records, and computes aggregated daily intakes. The server consults medical standard information indicating that a safe upper intake level for vitamin A is lower than 10000 IU.

The server computes a high risk level for vitamin A, a moderate risk level for zinc, and low or no risk for the other components. The server constructs a prompt sentence such as: “Given the following user supplement intake data: Vitamin A 10000 IU daily, Vitamin C 1000 mg daily, Vitamin D 2000 IU daily, and Zinc 50 mg daily, and given that the tolerable upper intake level for Vitamin A is 3000 IU per day and for Zinc is 40 mg per day, explain the potential long-term health risks caused by exceeding these upper limits and provide clear recommendations to reduce the risks, using language understandable by a non-expert user.”

The server encodes this prompt sentence into tokens and executes the generative AI model to obtain a response sentence describing, for example, possible effects of vitamin A overconsumption on liver function and bone health, and of zinc overconsumption on copper absorption. The server merges this explanation with numeric risk details and sends the resulting warning information to the terminal, which displays it with appropriate visual emphasis.

In another embodiment, the server uses multiple alternative model configurations. The server may use a smaller generative AI model for low-severity risks and a larger generative AI model for complex, high-severity scenarios, selecting between them based on computed severity and resource availability. The server may also vary the prompt structure, for example by including more detailed numeric context when the variance between actual intake and upper limit exceeds a threshold, or by omitting less relevant components to reduce prompt length. This adaptive prompt construction and model selection leads to improved computational efficiency and stability of generated outputs, which is a technical improvement beyond simply automating a human expert's drafting of recommendations.

In yet another embodiment, the server includes a monitoring module that measures processing time, memory consumption, and network traffic for each end-to-end evaluation and explanation cycle. The server can adjust criteria for invoking the generative AI model based on these measurements, such as raising severity thresholds when system load is high, in order to maintain service quality. This dynamic control of when and how the generative AI model is used contributes to overall system performance, reduces bandwidth usage by limiting large prompt and response messages under load, and maintains consistent response latency.

The server and terminal collectively improve computer technology by implementing a pipeline that (i) enforces early normalization and structuring of user input to reduce ambiguous or redundant processing, (ii) uses efficient aggregation on normalized data to compute risk levels, (iii) constructs prompt sentences programmatically from numeric context rather than relying on ad hoc textual descriptions, and (iv) selectively uses resource-intensive generative AI inference only when justified by risk severity. These design choices result in lower error rates in numeric-to-text mapping, higher throughput of evaluations per unit of computational resource, and more stable, policy-compliant natural language output from the generative AI model. The system therefore does not merely computerize a human decision process, but rather re-architects the internal data structures, processing order, and model invocation logic to achieve specific technical advantages in data management, computation, and communication in a networked computing environment.

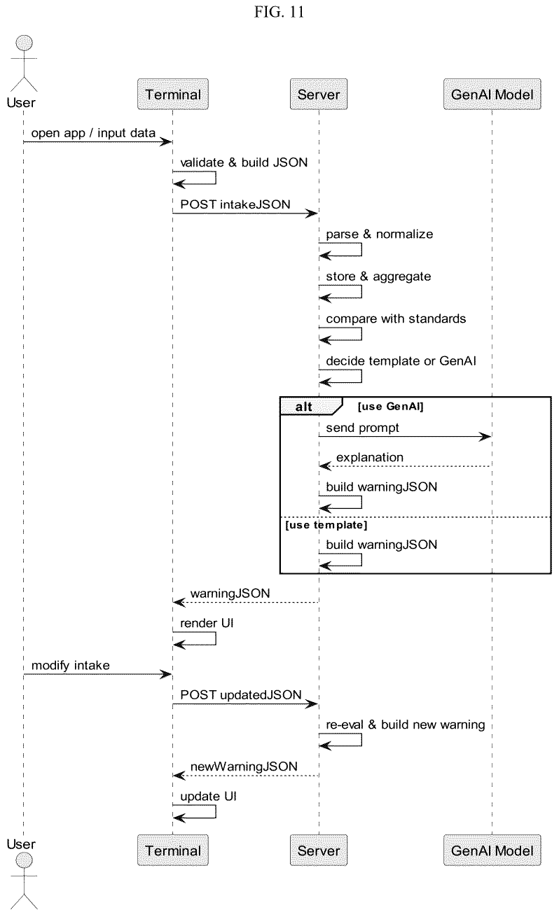

The following describes the processing flow using FIG. 11.

### Step 1:

The user operates the terminal to launch a supplement management application and open an intake entry screen. The terminal receives raw input from the user, including free-form text for supplement names, component names, numeric strings for intake amounts, unit selections, and frequency expressions. The terminal uses the input as data to construct an internal intake record object, mapping each UI field to a corresponding attribute. The terminal performs basic client-side validation by checking that required fields are non-empty and that numeric strings can be parsed into numbers; if invalid input is detected, the terminal displays error messages and requests correction. The output of this step is a validated internal intake record object in the terminal memory.

### Step 2:

The terminal converts the internal intake record object into structured information suitable for network transmission. The input to this step is the validated intake record object from Step 1.

The terminal normalizes some values, such as converting a user-entered frequency phrase (“once a day”) into a numeric frequency value (1.0) and mapping selected units (“mg”, “IU”) to standardized unit codes. The terminal then serializes the normalized object into a structured data representation, such as a JSON-like logical structure, using a serialization library. The terminal adds metadata fields including a user identifier, a timestamp, and an application version. The output of this step is a serialized structured intake information message ready to be sent over the network.

### Step 3:

The terminal transmits the structured intake information message to the server over a secure communication channel. The input to this step is the serialized message from Step 2. The terminal uses a network stack and an HTTP client implementation to open a secure connection, attach authentication tokens in request headers, and send the message as the body of an HTTP request. The terminal also sets timeout values and retry policies for transient failures. The output of this step is an HTTP request received by the server that contains the structured intake information in its payload.

### Step 4:

The server receives the HTTP request containing the structured intake information via a network interface and passes it to a server application. The input to this step is the raw HTTP request containing the serialized data. The server parses the request using a protocol handler, extracts the body, and uses a parser library to convert the serialized data into internal data structures, such as objects or records in memory. The server validates the data format by checking expected field names, data types, and presence of required fields. The server discards or flags malformed records. The output of this step is a normalized in-memory representation of the intake information, with basic format validity confirmed.

### Step 5:

The server performs content normalization to convert heterogeneous user input into canonical internal representations. The input to this step is the parsed intake information from Step 4.

The server uses predefined mapping tables and conversion rules to unify units (for example, converting all vitamin A values into a base unit), to align free-text component names to canonical component identifiers, and to standardize casing and whitespace. The server uses algorithms such as string normalization and table lookup to map user-entered labels to stored identifiers. The server may apply range checks and clamp unrealistic values if necessary. The output of this step is a fully normalized intake record, including canonical component identifiers, standardized units, and converted numeric intake amounts.

### Step 6:

The server stores the normalized intake record in a storage device for later aggregation and analysis. The input to this step is the normalized record from Step 5. The server computes a storage key combining the user identifier and timestamp and writes the record into a database table or collection dedicated to intake records. The server also updates indexes to allow efficient retrieval by user and by time period. The server may log the successful write operation for audit purposes. The output of this step is a persistent intake record stored in the database and accessible via indexed queries.

### Step 7:

The server retrieves, for a given user, historical intake records over a predetermined period in preparation for risk evaluation. The input to this step is a user identifier and a time range, obtained either from the latest intake submission or from stored user settings. The server issues database queries filtering records by user identifier and timestamp interval, using available indexes to limit scanned rows. The server loads matching records into memory as structured data collections grouped or groupable by component identifier. The output of this step is a set of normalized intake records for the user over the target period.

### Step 8:

The server aggregates intake amounts for each component over the predetermined period. The input to this step is the set of intake records from Step 7. The server iterates over each record, groups records by component identifier, and applies arithmetic operations such as summation to calculate total intake per component. The server then computes averages or other metrics such as daily or weekly intake by dividing by the number of days in the period or by other relevant factors. Because all records share canonical units and identifiers, the server performs these operations directly on numeric values without further conversions. The output of this step is an aggregated data structure that maps each component identifier to its computed intake metrics (for example, total daily, weekly, and monthly amounts).

### Step 9:

The server compares the aggregated intake metrics to stored medical standard information to compute risk levels and interaction risks. The input to this step is the aggregated component intake data from Step 8 and the medical standard information loaded from storage. The server, for each component, retrieves the upper intake limit and recommended intake amount from the medical standard information and performs numeric comparisons to determine whether the aggregated intake exceeds the limit and by what ratio or difference. The server assigns a risk level based on predefined thresholds, such as low, moderate, or high. The server then evaluates interaction rules that specify combinations of components and associated risk conditions; using Boolean logic on presence/absence flags and intake thresholds, the server determines if any interaction risks are triggered. The output of this step is a structured risk evaluation result containing, for each component and component combination, numeric comparisons, a risk level, and an interaction risk indicator where applicable.

### Step 10:

The server decides whether to use a template-based warning generation path or a generative-AI-based explanation path based on the computed risk levels and interaction risks. The input to this step is the structured risk evaluation result from Step 9 and configured severity thresholds. The server examines each component's risk level and interaction risk; if all risks are below a configured threshold, the server chooses the template-based path; if any risk is at or above the threshold, the server selects the generative-AI-based path. This decision is implemented as conditional logic on the aggregated risk data. The output of this step is a control decision indicating, for each warning instance or for the overall evaluation, which generation mode to apply.

### Step 11:

The server generates a template-based warning when the template-based path is selected. The input to this step is the structured risk evaluation result and the control decision from Step 10. The server inserts numeric values such as actual intake, upper limit, and risk level into predefined text templates, using string formatting operations. The server creates warning entries including component identifiers, danger categories, and short explanatory sentences derived from rules. The server assembles these entries into warning information structured data. The output of this step is template-based warning information ready for transmission to the terminal.

### Step 12:

The server constructs a prompt sentence for the generative AI model when the generative-AI-based explanation path is selected. The input to this step is the structured risk evaluation result and the control decision from Step 10. The server formats component identifiers, aggregated intake values, upper limits, and identified risks into a coherent natural language description by concatenating sentence fragments with embedded numeric values. The server also appends an instruction segment specifying the tone, level of detail, and target audience (for example, non-expert users). As one example, the server generates a prompt sentence such as: “Given the following user supplement intake data: Vitamin A 10000 IU daily, Vitamin C 1000 mg daily, Vitamin D 2000 IU daily, and Zinc 50 mg daily, and given that the tolerable upper intake level for Vitamin A is 3000 IU per day and for Zinc is 40 mg per day, explain the potential long-term health risks caused by exceeding these upper limits and provide clear recommendations to reduce the risks, using language understandable by a non-expert user.”

The output of this step is a complete prompt sentence string that encodes numeric and contextual data for the generative AI model.

### Step 13:

The server submits the prompt sentence to the generative AI model and obtains a response sentence. The input to this step is the prompt sentence from Step 12. The server tokenizes the sentence into token identifiers using a tokenizer associated with the generative AI model, sends the token sequence to a model execution engine running on suitable hardware, and initiates inference. The generative AI model processes the token sequence through multiple transformer layers, computing attention scores and updated hidden states to predict subsequent tokens according to a language modeling objective. The server receives generated tokens from the model until a termination condition is met and then decodes the tokens back into text. The server may apply truncation or filtering to the generated text. The output of this step is a response sentence that provides a natural language explanation of the identified risks and recommendations.

### Step 14:

The server constructs warning information that combines numerical risk data and generated explanations. The input to this step is the structured risk evaluation result from Step 9 and the response sentence from Step 13 (or the template-based text from Step 11 when applicable). The server builds a structured warning object containing, for each component and component combination with non-zero risk, the component identifier, numeric details (actual intake versus limit), danger category, and warning content. The warning content may include a portion of the response sentence or the entire response sentence, possibly segmented per component. The server then serializes this structured warning object into a response message suitable for network transmission. The output of this step is a serialized warning information message ready to be sent to the terminal.

### Step 15:

The server transmits the warning information message to the terminal over the network. The input to this step is the serialized message from Step 14. The server encapsulates the message in an HTTP or similar response, sets appropriate headers such as content type and status code, and sends the response via the network interface. The server may also log the transaction details, including user identifier, timestamp, and abbreviated risk summary. The output of this step is a network response received by the terminal that contains the warning information payload.

### Step 16:

The terminal receives the warning information message and prepares it for display. The input to this step is the network response containing the serialized warning payload from Step 15.

The terminal uses a client-side parser to convert the serialized content into local data structures, such as lists of warning items, each with component identifier, numeric details, danger category, and warning content. The terminal evaluates the danger categories to determine how to visually prioritize warnings, such as sorting by severity. The output of this step is an in-memory warning list ready for rendering on the user interface.

### Step 17:

The terminal displays the warning information to the user with visual distinction according to danger categories. The input to this step is the warning list from Step 16. The terminal renders each warning item in a user interface component, applying visual styles such as color, icons, or layout changes based on danger category (for example, red for high risk, yellow for moderate risk). The terminal displays the explanation text in a scrollable area and numeric details in structured form, such as “intake 10000 IU vs upper limit 3000 IU.” The terminal also presents interactive elements, such as buttons or editable fields, that allow the user to modify the previously entered intake information. The output of this step is a visual presentation of the warnings and an interactive interface enabling user corrections.

### Step 18:

The user reviews the warning information and decides whether to change the supplement regimen. The input to this step is the visual display from Step 17. The user may decide, for example, to reduce the intake amount of vitamin A or decrease the frequency of zinc intake.

The user enters the changes using the terminal's input controls, resulting in updated intake values in the terminal's internal data model. The output of this step is a modified intake record object stored in the terminal's memory.

### Step 19:

The terminal processes the modified intake record object and resubmits it to the server for reevaluation. The input to this step is the modified record from Step 18. The terminal repeats the serialization and normalization operations similar to Steps 2 and 3: it recomputes frequency values if they changed, revalidates numeric fields, and generates a new structured intake information message. The terminal then transmits this message to the server via the secure network connection. The output of this step is a new HTTP request containing updated intake information reaching the server.

### Step 20:

The server receives the updated intake information and performs incremental reevaluation.

The input to this step is the updated structured intake message from Step 19. The server parses and normalizes the new record (as in Steps 4 and 5), updates the stored intake records (as in Step 6), and recomputes aggregated intake metrics for the affected components (as in Step 8).

The server then repeats the risk comparison (Step 9), generation mode decision (Step 10), and warning generation (Steps 11-14) for the updated data. Because the server operates on normalized stored records and can restrict aggregation to the modified time windows or components, the server reduces redundant computation. The output of this step is updated warning information reflecting the modified intake.

### Step 21:

The terminal receives the updated warning information and presents the new results to the user. The input to this step is the updated warning message from Step 20. The terminal parses the message, updates the displayed danger categories and explanations, and may show that certain risks have decreased or been eliminated. The user can see the technical effect of the modifications, and the iterative loop can continue as necessary. The output of this step is an updated user interface state in which current risks are accurately reflected based on the latest server-side computation and generative AI model explanation.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented health management systems that track nutritional supplement intake typically rely on static rule sets or manually defined thresholds to evaluate potential health risks. These systems are often implemented as straightforward database applications that simply aggregate per-product intake amounts and compare them to fixed reference values. As a result, such systems suffer from several technical shortcomings.

First, existing systems are not architected to efficiently integrate heterogeneous intake data streams, such as electronic transaction records, user-entered intake schedules, and time-varying health examination information, into a unified data structure suitable for dynamic risk evaluation. The lack of structured, time-segmented data models for nutrient intake across multiple products leads to inefficient queries, redundant calculations, and increased processing latency, particularly when analyzing long-term intake patterns for a large number of users.

Second, when generative AI models are used in a naïve way, they are often invoked with unstructured or poorly constrained textual input, which causes unpredictable responses, increases token size, and complicates downstream parsing. Conventional systems do not provide a systematic mechanism for constructing machine-oriented prompt sentences that embed structured, normalized intake data and time-based aggregates. As a consequence, server-side components must perform extensive ad hoc post-processing of the model output, which increases computational overhead and reduces reliability of the evaluation pipeline.

Third, existing architectures do not provide an efficient feedback loop between updated user behavior and the generative AI-based risk evaluation. When a user changes intake conditions, traditional systems either require complete recomputation from raw history or rely on approximate updates that do not consistently propagate into the AI model input. This lack of incremental recalculation and dynamic prompt updating degrades the responsiveness and scalability of the system, and may result in outdated or inconsistent risk assessments being delivered to user terminals.

Fourth, conventional notification mechanisms are not optimized to handle prioritized risk information generated by AI models. Typical implementations deliver all risk details to the terminal without filtering or structuring, which can overwhelm users and also impose unnecessary communication and processing load on both server and client devices. There is no integrated mechanism by which a server can perform priority assignment on AI-derived risk levels, generate concise warnings only for high-priority items, and persist detailed evaluations in a form that is efficiently retrievable when needed.

Accordingly, there is a need for an improved computer-implemented system that (i) acquires and normalizes electronic transaction and intake data for multiple nutritional products, (ii) structures nutrient-level intake information on a time-segment basis, (iii) generates machine-readable prompt sentences that efficiently drive a generative AI model to produce stable and structured risk assessments, and (iv) dynamically updates both the underlying aggregates and the AI prompts in response to user behavior changes, while (v) generating prioritized, concise warnings for delivery to user terminals. Such a system should improve the overall performance, scalability, and reliability of the health risk evaluation pipeline executed by the server and reduce the computational and communication burden associated with AI-based risk analysis.

The specific processing by the specific processing unit 290 of the data processing device 12 in Application Example 1 is realized by the following means.

The present invention provides a server comprising a processor and a communication interface configured to acquire, from a user terminal via the communication interface, electronic transaction purchase information and intake information relating to a plurality of nutritional products; to normalize and store the purchase information as intake history data including nutrient-level component information in a storage resource; to aggregate, for each user, a daily intake amount and a cumulative intake amount for each nutrient component on a plurality of time segments based on the intake history data; to construct, from the aggregated nutrient-level time-segment data and from health examination information received from the user terminal, a structured prompt sentence that encodes the data in a machine-oriented format; to supply the structured prompt sentence to a generative AI model as input and to obtain, from the generative AI model, structured evaluation information indicating at least a risk level and a recommended action for each nutrient component; to perform priority assignment processing on the evaluation information so as to select nutrient components or nutritional products whose risk level satisfies a predetermined priority condition; to generate concise warning information only for the selected nutrient components or nutritional products and to transmit the concise warning information to the user terminal via the communication interface for display by a notification function of the user terminal; and to recalculate updated nutrient intake aggregates and dynamically update the structured prompt sentence when the intake information is changed by the user, and to cause the generative AI model to perform reevaluation based on the updated prompt sentence. This enables improved computer operation for health risk evaluation by structuring heterogeneous intake and health data into time-segmented nutrient-level aggregates suitable for efficient AI prompting, by reducing model input size and post-processing complexity through machine-oriented prompt construction, by providing an incremental update mechanism that maintains consistency between user behavior changes and AI-based risk assessments, and by optimizing communication and client-side processing through prioritized, concise warning delivery while preserving detailed evaluation data in the server-side storage resource for on-demand retrieval.

The term “system” refers to a combination of hardware and software components including at least one server-side processor and at least one communication interface that cooperatively perform the functions described in the claims.

The term “processor” refers to one or more hardware processing units, such as a central processing unit or a processing core, configured to execute instructions that cause the system to perform data acquisition, aggregation, prompt generation, model interaction, evaluation, and notification processing.

The term “communication device” refers to one or more hardware and software communication interfaces, such as network interface controllers and associated protocol stacks, configured to transmit and receive data between the system and external devices including user terminal devices and external services.

The term “user terminal device” refers to an electronic device operated by a user, such as a mobile device, a handheld device, or a general-purpose computing device, that is capable of communicating with the system, providing user input, and displaying information including warnings and recommendations.

The term “electronic transaction” refers to a purchase or acquisition of goods or services that is executed by electronic means over a communication network using an electronic device.

The term “purchase information” refers to data representing details of an electronic transaction for a nutritional product, including at least an identifier of the product, a quantity, and a date of purchase, and optionally including additional attributes such as unit amount or source of purchase.

The term “intake information” refers to data indicating how a user consumes a nutritional product, including at least an amount per intake, a frequency of intake, and a duration or schedule of intake.

The term “nutritional product” refers to an ingestible composition intended to supplement diet or nutrition, including supplements that contain one or more nutrient components such as vitamins, minerals, or other active substances.

The term “nutrient component” refers to a chemical or biological component contained in a nutritional product, such as a vitamin, a mineral, or another nutritionally relevant substance, for which an intake amount can be quantified.

The term “intake history information” refers to stored data that associates a user with nutritional products, nutrient components, and corresponding intake information over time, including purchase events and consumption patterns.

The term “storage resource” refers to one or more logical or physical data stores, such as database systems or memory resources, configured to retain purchase information, intake history information, evaluation information, and warning information.

The term “aggregate” refers to a computational operation that combines multiple data elements, such as individual intake records, into summarized values such as a daily intake amount, a cumulative intake amount, or a time-based statistic.

The term “daily intake amount” refers to a calculated quantity of a nutrient component that a user consumes in a given day, derived from intake information and product composition information.

The term “cumulative intake amount” refers to a calculated quantity representing the sum of daily intake amounts of a nutrient component over a specified period.

The term “time segment” refers to a defined unit or interval of time, such as a day, a week, or a month, used to organize and index intake amounts and evaluation data.

The term “health examination information” refers to medical or health-related data obtained from or on behalf of a user, such as test results, measurements, or diagnoses, that is used as input to the risk evaluation.

The term “prompt sentence” refers to a structured set of textual instructions and embedded data, prepared by the processor, that is supplied as input to a generative AI model to request generation of evaluation information in a specified manner.

The term “structured prompt sentence” refers to a prompt sentence that includes intake and health-related data organized in a machine-oriented format, such as key-value pairs or structured text, to enable deterministic and efficient processing by a generative AI model.

The term “generative AI model” refers to a machine learning model, such as a large language model or other generative model, configured to generate output data including text or structured data in response to input data including a prompt sentence.

The term “evaluation information” refers to output data generated by the generative AI model based on the prompt sentence, including at least risk levels, explanations, or recommended actions related to nutrient components or nutritional products.

The term “risk level” refers to a categorization value, such as low, moderate, or high, indicating a degree of potential adverse health impact associated with the intake of a nutrient component or nutritional product.

The term “recommended action” refers to guidance generated by the generative AI model or derived from its output, indicating a suggested change or behavior for the user, such as adjusting intake or consulting a professional.

The term “priority assignment processing” refers to a computation performed by the processor to assign priority values to elements of evaluation information, such as nutrient components or nutritional products, based on their risk levels or other criteria.

The term “concise warning information” refers to a shortened or summarized representation of risk-related information, generated by the processor, that is suitable for quick display on a user terminal device and that focuses on high-priority risks.

The term “warning information” refers to data indicating that a potential health risk has been identified for a user, including at least an indication of the associated nutrient component or nutritional product and a representation of the severity or nature of the risk.

The term “recommendation information” refers to data that presents one or more suggestions or proposals to the user concerning the intake of nutritional products, based on evaluation information from the generative AI model.

The term “notification function” refers to functionality of a user terminal device that provides visual, auditory, or tactile alerts to a user, such as system-level notifications or in-application alerts.

The term “dynamic update” refers to a process in which stored data, aggregates, or prompt sentences are recalculated or modified in response to newly received information, such as changed intake conditions, without reconstructing all prior computations from scratch.

The term “reevaluation” refers to a repeated execution of a risk assessment process by the generative AI model using updated prompt sentences and updated underlying data, resulting in updated evaluation information.

The term “structured data” refers to data that is organized according to a defined format or schema, such as tables, arrays, or labeled fields, enabling direct and predictable interpretation by computer processes.

The term “intake conditions” refers to parameters that describe how a user currently consumes nutritional products, including intake amounts, timing, frequency, and any changes to these parameters over time.

In one embodiment, a server cooperates with at least one terminal and at least one communication network to implement the claimed system. The server includes a hardware processor, a memory device, a non-transitory storage device, and a network interface controller. The terminal includes a hardware processor, a display device, an input device, a memory device, a non-transitory storage device, and a wireless or wired communication interface. The user operates the terminal to perform electronic transactions for nutritional products, to provide intake conditions, and to view warnings and recommendations.

The server executes program modules that are stored in the non-transitory storage device and loaded into the memory device. The server executes operating system software, such as a general-purpose server operating system, and application software implemented, for example, in a scripting language or a compiled language. The server optionally executes a web application framework, such as an HTTP server framework, and a database management system such as a document-oriented database system or a key-value store. The server communicates with an external generative AI model endpoint via a network interface using a secure communication protocol such as HTTPS.

The terminal executes an operating system, such as a mobile operating system, and an application program for nutritional supplement management. The terminal application includes modules for electronic transaction monitoring, user input management, local data caching, and notification display. The terminal acquires purchase information from electronic transaction services via an application programming interface or by parsing electronic receipts, and sends structured intake and purchase information to the server using a secure network connection.

The server stores purchase information and intake information in a storage resource. The storage resource includes a database that stores, for each user, a plurality of records representing nutritional products, nutrient components, and intake conditions. The server represents each record as a structured data object including at least a product identifier, a nutrient component identifier, an amount per unit, an intake frequency, an intake start date, and a purchase date. The server additionally maintains an aggregate table or collection in which daily intake amounts and cumulative intake amounts are stored on a per-nutrient-component, per-time-segment basis for each user.

The server uses a data structure in which a time segment is stored as a discrete index, such as a day index, and an aggregate intake amount for each nutrient component is stored in association with the time segment. The server uses an index over user identifiers and nutrient component identifiers to accelerate aggregation queries. The server thus reduces the number of database read operations and computations required to obtain a long-term intake pattern by reading pre-computed daily and cumulative values, instead of recomputing all values from raw purchase and intake records. This data structure improves computational efficiency and reduces latency of risk evaluation for users with long histories and many nutritional products.

The server constructs a prompt sentence for a generative AI model by embedding structured intake and health examination information into a machine-oriented textual representation. The server uses a prompt-construction module that formats nutrient-level aggregates into a deterministic template. The server, for example, generates a prompt sentence of the following form:

“Using the following structured data for a single user's nutritional supplement intake, identify potential long-term health risks. For each nutrient (such as vitamin D, vitamin A, iron, calcium), determine whether the intake is within safe long-term limits, slightly above typical recommendations, or significantly above recommended upper levels. Consider total daily intake from all supplements and approximate duration of use. Return a list of entries with: nutrient, risk_level (low, moderate, high), explanation, and recommendation. Intake data: [vitamin D: daily 5000 IU for 180 days from supplement A and supplement B; vitamin A: daily 8000 IU for 90 days; iron: daily 30 mg for 120 days].”

In another embodiment, the server generates a shorter operational prompt sentence such as:

“Analyze the user's nutritional supplement purchase and intake history and evaluate possible long-term health risks, including overdoses and harmful combinations. Then generate a concise warning message suitable for display on a smartphone, limited to 200 characters, that summarizes the most important risk.”

The server supplies such prompt sentences to a generative AI model implemented, for example, as a transformer-based neural network. The generative AI model includes a plurality of layers, each layer including multi-head self-attention sublayers and feed-forward sublayers.

The model uses a token embedding module that maps input text tokens to vector representations, positional encodings that represent token positions in the prompt sentence, and attention mechanisms that compute context-aware representations of each token. The model is trained on a large corpus of text using supervised or semi-supervised learning. The model uses a loss function, such as a cross-entropy loss, which measures a difference between predicted token distributions and ground-truth tokens. During training, the model updates its weights using a gradient descent-based algorithm, such as stochastic gradient descent or an adaptive optimizer. The model optionally uses regularization techniques, such as dropout, to prevent overfitting.

The server, in some embodiments, uses a fine-tuning dataset containing de-identified nutritional supplement risk examples to adjust the generative AI model to the risk-evaluation domain. The server uses a training pipeline that constructs input-output pairs consisting of structured prompt sentences and desired structured responses including nutrient risk levels and recommendations. The server computes gradients of the loss function with respect to network parameters and updates the weights of the model through iterative training epochs.

By using structured prompts and supervised fine-tuning, the server causes the generative AI model to produce outputs that are easier to parse and that have stable formats, thereby reducing post-processing complexity and error rates.

The server processes the generative AI model output with a parsing module. The server constrains the output by including explicit formatting instructions in the prompt sentence, such as “Return a list of entries with: nutrient, risk_level, explanation, recommendation.” The server then tokenizes the output and extracts fields using rule-based patterns or a lightweight parser. By aligning the structure of the prompt sentence with the expected structure of the response, the server reduces ambiguity in the model output and improves parsing accuracy.

This cooperation between data structuring and prompt design leads to an improvement in the overall reliability and speed of the risk evaluation pipeline executed on the server hardware.

The server applies a priority assignment algorithm to the evaluation information output by the generative AI model. The server, for example, maps risk levels “low,” “moderate,” and “high” to numeric scores and computes an overall priority score per nutrient component and per nutritional product. The server may apply weighting factors based on duration of intake, daily dose relative to reference values, and user-specific health examination information. The server then selects only those nutrient components or products whose priority score exceeds a threshold. The server generates “concise warning information” for these selected items, such as:

“Excessive vitamin D intake detected. Please reduce total daily intake or consult a healthcare professional.”

By limiting warnings to high-priority items and by compressing them into short messages, the server reduces the volume of data transmitted to the terminal, improves the notification relevance for the user, and reduces both server-side and client-side processing overhead.

The server stores the detailed evaluation information in the storage resource, including the full explanation and the recommendations generated by the generative AI model. The server associates each concise warning message with a detailed record. When the user requests more information via the terminal, the server retrieves the associated record and transmits the detailed explanation to the terminal. This two-level structure (concise warning and detailed record) reduces network traffic in normal operation and avoids overloading the user interface, but still allows full access to detailed information when requested.

The terminal presents warnings and recommendations using a notification function. The terminal receives concise warning information via a push notification service or a polling mechanism and displays it as a system-level notification or an in-application alert. When the user taps the notification, the terminal requests the corresponding detailed evaluation from the server and displays the detailed risk levels and recommendations in a dedicated screen. The terminal uses graphical user interface components to show, for example, lists of nutrients, risk levels, colored indicators, and textual explanations. The terminal, in some embodiments, stores a local cache of recent warnings in local storage to allow quick access and offline viewing.

The user adjusts intake conditions in response to the warnings and recommendations. The user modifies, through the terminal application, parameters such as number of units per day, intake days per week, or discontinuation of a nutritional product. The terminal sends updated intake conditions to the server. The server updates the intake history information and recomputes daily and cumulative intake amounts for the relevant nutrient components. The server then regenerates a prompt sentence reflecting the updated intake pattern and resubmits the prompt to the generative AI model. In this way, the system forms a closed loop in which user behavior changes cause incremental recalculation, refined AI input, and updated risk assessment.

The server, by using pre-aggregated time-segment data structures and by dynamically updating only affected aggregates, avoids recomputing all historical intake amounts from scratch whenever the user modifies intake conditions. The server updates only the records corresponding to time segments affected by the change. This approach improves computational efficiency, reduces response time, and enhances scalability when many users and long histories are present.

The system provides technical improvements over conventional systems in several ways. The server improves data management by organizing nutrient intake data into time-segmented, user-indexed, structured records that support efficient aggregation and retrieval operations.

The server improves computational efficiency by precomputing and maintaining daily and cumulative intake amounts and by limiting reevaluation computations to affected segments and high-priority risks. The server improves communication efficiency by generating concise, prioritized warnings for delivery to the terminal while storing detailed data in the storage resource. The server improves overall system accuracy and stability by using carefully designed structured prompt sentences that constrain the generative AI model's output format and content, thus reducing parsing errors and inconsistent responses.

The server also uses a non-conventional combination of rule-based processing and generative AI processing. The server does not merely automate human judgment. Instead, the server establishes a technical pipeline in which domain-specific normalization, aggregation, and prompt construction provide a structured representation that exploits the underlying transformer network's capability to model complex interactions between nutrient components and time patterns. The server's rule-based priority assignment and thresholding further refine and filter AI outputs to produce machine-efficient, reliable results. This non-traditional interaction between deterministic aggregation logic and probabilistic generative modeling yields a computational process that is not achievable when a human manually reviews raw purchase records or manually queries static reference tables.

In another embodiment, the server executes a locally deployed generative AI model instead of an external service. The server loads a pretrained transformer-based neural network model from the storage device into memory and performs inference using local hardware, such as a graphics processing unit or a tensor processing unit. The server uses optimized matrix multiplication libraries to accelerate attention and feed-forward computations. This embodiment reduces network latency and external dependency and allows further customization of the model. The server may use quantization or model distillation techniques to reduce memory footprint and inference time.

In another embodiment, the server uses different neural network architectures, such as encoder-only or encoder-decoder transformers, or a recurrent neural network with attention, provided that the model can accept prompt sentences and output structured evaluation information. The server may use alternative loss functions, regularization strategies, or training schedules to tailor the generative AI model to various user populations or product sets.

In a further embodiment, the server uses additional features as input to the generative AI model. The server includes, in the prompt sentence, user demographic attributes, history of prior warnings, and adherence patterns inferred from time gaps between purchases and intake logs. The server may also include statistical features such as variance in daily intake amount over a period, which the generative AI model may use to detect irregular consumption patterns. By including such derived features in a structured prompt, the server enables improved detection of complex risk patterns and enhances prediction accuracy beyond simple threshold-based systems.

The system can be applied to various use cases in which long-term intake of multiple nutritional products must be monitored and evaluated. The server can be used in personal health management, occupational health programs, or institutional wellness services. The terminal can be implemented in a smartphone, a tablet, or other portable device. The described architecture, with its structured data organization, dynamic prompt generation, and priority-based warning transmission, is not limited to any specific combination of hardware or software brands and can be implemented using various general-purpose computing devices and databases, as long as the described functional modules and data flows are realized.

Through these embodiments, the server, the terminal, and the user cooperate to implement a system that uses a generative AI model and structured prompt sentences not merely to automate a human risk evaluation task, but to improve internal computer operations related to data structuring, computation, communication, and AI interaction, thereby achieving faster, more accurate, and more resource-efficient health risk assessments.

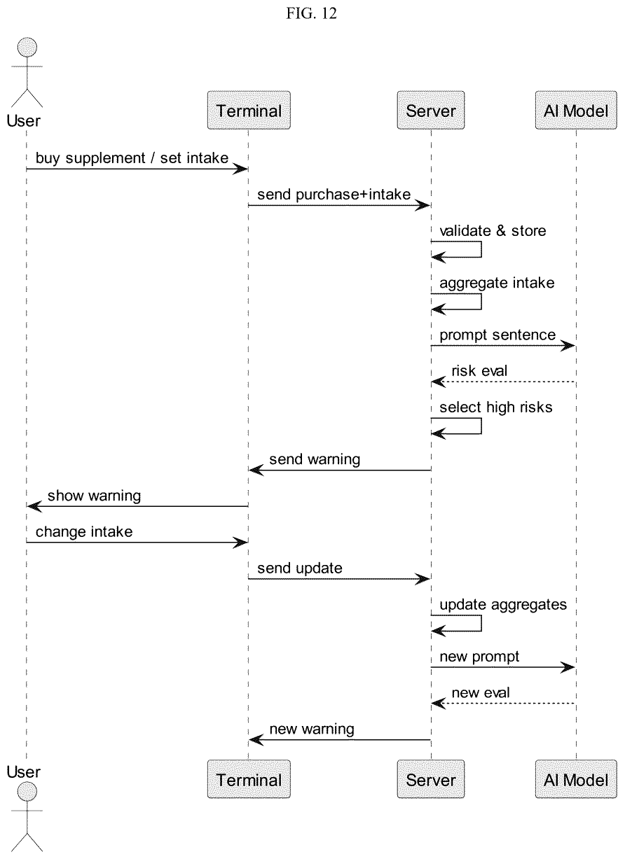

The following describes the processing flow using FIG. 12.

### Step 1:

The user performs an electronic transaction for a nutritional product using the terminal.

The terminal receives, as input, transaction data from an electronic commerce service, including at least product identifier, quantity, unit size, purchase date, and price.

The terminal parses email receipts or application programming interface responses, extracts relevant fields using string processing and structured data parsers, and converts the raw transaction data into a normalized purchase record.

The terminal outputs a structured purchase record in an internal format with fields such as user identifier, product identifier, quantity, unit amount, and purchase timestamp.

### Step 2:

The terminal sends the structured purchase record to the server over a communication network.

The terminal uses, as input, the normalized purchase record created in Step 1 and an authentication token associated with the user.

The terminal opens a secure connection, constructs a request message containing the purchase record in a structured payload, and transmits the message to a predefined server endpoint.

The terminal outputs a network request that the server can receive and process for storage and later aggregation.

### Step 3:

The server receives and validates the purchase record from the terminal.

The server uses, as input, the network request payload containing the structured purchase record.

The server parses the payload, checks for required fields, verifies value ranges (for example, that quantity is positive), and ensures that identifiers match known formats.

The server outputs either a validated purchase record ready for storage or an error response if validation fails.

### Step 4:

The server enriches the purchase record with product composition information and stores it in a storage resource.

The server uses, as input, the validated purchase record and a reference data store containing nutritional product metadata (for example, ingredient lists and per-unit nutrient amounts).

The server performs a lookup using the product identifier, retrieves nutrient component information (such as vitamin D amount per unit, iron amount per unit), merges this information into the purchase record, and writes the enriched record into a user-specific collection in a database.

The server outputs an intake history entry that associates the user with the nutritional product, nutrient components, and purchase context.

### Step 5:

The user specifies or adjusts intake conditions for each nutritional product using the terminal.

The terminal uses, as input, the list of products and composition data retrieved from the server and user operations through input controls (for example, text fields and selection widgets).

The terminal allows the user to set parameters such as units per day, days per week, intended start date, and optional end date, then validates these values locally.

The terminal outputs updated intake condition records and sends them to the server as structured user configuration data.

### Step 6:

The server stores intake conditions and links them to intake history entries.

The server uses, as input, the intake condition records received from the terminal and the existing intake history entries for the same user and products.

The server associates each intake condition with the corresponding product and nutrient components, writes the records into configuration collections, and maintains relationships between history, composition, and user-defined schedules.

The server outputs a consistent internal representation in which purchase events, composition data, and intake conditions are linked for subsequent computation.

### Step 7:

The server aggregates nutrient intake per time segment for each user.

The server uses, as input, the intake history entries, composition data, and intake conditions associated with a user.

The server computes, for each time segment (for example, each day), an expected intake amount per nutrient component by multiplying units per day by amount per unit and summing across all active products; the server also integrates over multiple days to produce cumulative intake values.

The server outputs a set of aggregate records containing, for each user, for each nutrient, and for each time segment, a daily intake amount and a cumulative intake amount.

### Step 8:

The server constructs a structured prompt sentence for the generative AI model.

The server uses, as input, the aggregate records from Step 7 and health examination information obtained from the user via the terminal (for example, lab values or medical flags).

The server formats the data into a deterministic textual template, listing nutrient names, daily intake amounts, cumulative exposure periods, and relevant health indicators, and embeds explicit instructions about the required output structure (for example, specifying that the output must contain nutrient, risk_level, explanation, and recommendation).

The server outputs a complete prompt sentence that encodes the structured intake and health data in a machine-oriented format suitable for the generative AI model.

### Step 9:

The server sends the prompt sentence to the generative AI model and receives evaluation information.

The server uses, as input, the constructed prompt sentence from Step 8 and configuration parameters for the generative AI model (such as model name, maximum output length, and sampling settings).

The server transmits the prompt sentence to the generative AI model via an application programming interface, waits for the model to perform its internal transformer-based computations, and receives a textual response containing risk descriptions and recommendations.

The server outputs a raw model response that contains unparsed evaluation information for the user's nutrient intake.

### Step 10:

The server parses and structures the evaluation information generated by the generative AI model.

The server uses, as input, the raw textual response received in Step 9 and the expected response format specified in the prompt sentence.

The server identifies segments of the response corresponding to nutrient names, risk levels, explanations, and recommendations, applies rule-based parsing or simple pattern matching to split and label data, and converts the parsed elements into structured entries.

The server outputs a structured evaluation dataset in which each item is associated with a nutrient component and contains at least a risk_level, an explanation, and a recommended action.

### Step 11:

The server assigns priorities to risks and selects items for concise warning messages.

The server uses, as input, the structured evaluation dataset from Step 10 and predefined mapping rules from risk levels to priority scores, optionally including weights based on duration of intake and deviation from reference values.

The server computes a priority score for each nutrient or product, compares the scores to a predetermined threshold, and filters out items that do not meet the threshold; the server may also sort items by priority.

The server outputs a reduced list of high-priority risk items that will be used to generate concise warnings.

### Step 12:

The server generates concise warning information and stores detailed evaluation records.

The server uses, as input, the filtered list of high-priority risk items and the full structured evaluation dataset.

The server synthesizes short text messages summarizing the most important risks (for example, “Excessive vitamin D intake detected. Please reduce your dosage.”) and associates each concise message with the corresponding full evaluation entry in the storage resource; the server also saves timestamps and references to the underlying intake data used for the evaluation.

The server outputs concise warning records ready for transmission to the terminal and detailed evaluation records stored for later retrieval.

### Step 13:

The server transmits concise warning information to the terminal.

The server uses, as input, the concise warning records from Step 12 and addressing information for the user's terminal (for example, a device identifier or notification token).

The server packages each concise warning into a notification payload, attaches metadata such as a warning identifier, and sends the payload to the terminal using a push notification service or an application-level messaging protocol.

The server outputs network messages that cause the terminal to receive and display risk-related notifications.

### Step 14:

The terminal receives and presents concise warnings to the user.

The terminal uses, as input, the notification payloads sent by the server, which contain concise warning text and warning identifiers.

The terminal decodes the payload, invokes its notification function to display the warning to the user, and, upon user interaction, may request detailed evaluation information from the server using the warning identifier.

The terminal outputs a visual or auditory alert to the user and, when requested, a user interface screen that contains more detailed risk information retrieved from the server.

### Step 15:

The user reviews warnings and modifies intake conditions via the terminal.

The user uses, as input, the concise warnings and optional detailed evaluations displayed on the terminal's screen.

The user decides whether to change the number of units consumed per day, pause a nutritional product, or alter frequency, and enters updated values into the terminal application; the terminal validates and transmits the new conditions to the server.

The user outputs updated intake parameters that trigger further processing and reevaluation by the server.

### Step 16:

The server updates aggregates and initiates reevaluation when intake conditions change.

The server uses, as input, the updated intake conditions received from the terminal and the existing aggregate records and history data.

The server recalculates daily and cumulative intake amounts only for affected nutrient components and time segments, updates the aggregate records, reconstructs a new prompt sentence reflecting the modified intake pattern, and submits the updated prompt sentence to the generative AI model to obtain a new evaluation.

The server outputs refreshed evaluation information and, if necessary, new warning information that reflects the user's changed behavior, thereby maintaining up-to-date risk assessment with reduced computational overhead.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented systems for nutritional supplement recommendation typically rely on static rule sets, fixed threshold logic, or manually designed questionnaires executed by generic processors. In such systems, health checkup results and supplement intake histories are processed in a relatively shallow manner: numeric values are compared to preset ranges, and pre-authored recommendation templates are selected based on simple conditional branches. As a result, these systems exhibit several technical limitations from a computing standpoint.

First, conventional systems are not architected to dynamically construct and supply rich, context-aware input representations suitable for modern generative AI models. Raw health data and supplement histories are often passed as simple parameter lists or are not integrated at all into generative models. Without automatic feature generation and structured prompt construction, the processing pipeline cannot fully exploit the expressive capacity of a generative AI model, leading to suboptimal use of computing resources and reduced accuracy of the generated outputs.

Second, conventional architectures lack a well-defined mechanism to transform heterogeneous medical and behavioral data—such as biometric time-series, laboratory results, emotional state indicators, and long-term supplement histories—into unified, machine-readable prompt sentences for a generative AI model. The absence of this transformation layer places a burden on downstream computation, causing unnecessary complexity in model-side logic and irregular utilization of processor, memory, and communication resources.

Third, when generative AI models are used naively, their outputs are returned to users as unstructured natural language without further machine-level refinement. This output format is difficult for the system to validate, constrain, or combine with pre-stored reference criteria. As a consequence, the processor cannot reliably convert free-text recommendations into structured data suitable for deterministic rule processing, database storage, or longitudinal risk computation. This leads to inefficiencies, such as repeated parsing, ad hoc string processing, and increased latency, and may also degrade the reliability of the generated recommendations.

Fourth, prior systems usually treat biometric information and emotional state information as separate or optional inputs, rather than integral components of the computational pipeline.

They do not systematically incorporate continuous time-series biometric data and emotional state data into the generative AI prompts, nor do they support iterative updating of risk evaluations and recommendations as new data streams are ingested. This results in underutilization of available sensor data and a failure to provide technically improved, dynamically adaptive inference over time.

Moreover, there is insufficient integration between secure communication, feature normalization, prompt generation, model inference, response parsing, and rule-based post-processing as a cohesive, processor-executed workflow. Existing implementations do not clearly define, at the processor level, how encrypted data is obtained from terminals, how normalized feature data is generated, how prompt sentences are systematically constructed, and how structured outputs and reference rule sets are combined to refine generative AI outputs. This fragmented design prevents optimization of the end-to-end data flow and introduces redundant computation and network overhead.

Accordingly, there is a need for a computer-implemented system and processing architecture that: (i) automatically transforms encrypted health-related data received from a terminal into normalized feature data; (ii) constructs structured, context-rich prompt sentences for a generative AI model; (iii) converts the model's natural-language responses into structured data; and (iv) applies programmable rule processing to correct and refine the model outputs. Such a system should efficiently incorporate continuous biometric time-series and emotional state information into the computational pipeline, thereby improving the technical performance, reliability, and adaptability of the computer system that generates personalized supplement recommendations and health risk evaluations.

The specific processing by the specific processing unit 290 of the data processing device 12 in Example 2 is realized by the following means.

The present invention provides a server comprising a processor and a memory storing instructions that, when executed by the processor, cause the processor to receive, from a terminal, body information of a user and intake history information of the user; to acquire the body information and the intake history information from the terminal via an encrypted communication scheme, to normalize the body information, to calculate index values based on the body information, and to generate feature data based on the body information and the intake history information; to generate a description text including the feature data and the intake history information, and to create a prompt sentence including the description text; to input the prompt sentence into a generative artificial intelligence model and obtain, from the generative artificial intelligence model, a response text including an evaluation result of a potential health risk associated with long-term intake of nutritional supplements and including nutritional supplements recommended for intake and nutritional supplements not recommended for intake for the user; to extract, from the response text, identification information of the nutritional supplements, classification information of the nutritional supplements, recommended intake amounts, and caution information, and to generate structured data based on an extraction result; to execute rule processing on the structured data based on reference information stored in advance, to correct contents of the response text of the generative artificial intelligence model, and to determine final recommended nutritional supplements for intake and final non-recommended nutritional supplements for intake; and to generate warning information and explanation information for the user based on the evaluation result of the potential health risk and transmit the warning information and the explanation information to the terminal. This enables a computer-centric improvement in which the server automatically converts heterogeneous encrypted health inputs into normalized feature data, constructs context-rich prompt sentences for the generative AI model, transforms unstructured model outputs into machine-usable structured data, and applies deterministic rule processing to refine and stabilize recommendations, thereby enhancing computational efficiency, consistency, and adaptability of health risk evaluation and supplement recommendation across time, including when continuous biometric information and emotional state information are incorporated into the same processing pipeline.

The term “system” refers to an integrated combination of hardware resources and software resources configured to execute one or more processing operations described in the claims, including at least one processor, a memory, communication interfaces, and associated programs.

The term “processor” refers to one or more hardware computation units, such as a central processing unit or an accelerator, configured to execute instructions to perform data acquisition, data transformation, model inference, rule processing, and communication control.

The term “terminal” refers to an information processing device operated by a user, such as a mobile device, a portable device, or a general-purpose computer, configured to present screens, receive user input, and transmit and receive data to and from the server via a communication network.

The term “user” refers to a person whose body information, intake history information, biometric information, or emotional state information is acquired and processed by the system in order to evaluate health risks and generate nutritional supplement recommendations.

The term “body information” refers to health-related numerical values or categorical values concerning a physical state of the user, including, for example, measurement values from health checkups, laboratory test results, physical measurements, and other physiological indicators.

The term “intake history information” refers to information indicating past or current intake behavior of the user regarding nutritional supplements or related substances, including types of supplements, intake amounts, intake frequency, and intake duration.

The term “encrypted communication scheme” refers to a communication method using an encryption protocol that converts transmitted data into a format that is not directly interpretable by a third party, and that allows secure transfer of body information and intake history information between the terminal and the server.

The term “normalize the body information” refers to processing operations that convert raw body information into standardized numerical forms, including unit conversion, range scaling, and format unification, so that the information can be consistently used as input for subsequent computational steps.

The term “index values” refers to derived numerical indicators calculated from one or more items of body information, such as composite health scores, ratios, or diagnostic risk indices that summarize or emphasize aspects of the user's health status.

The term “feature data” refers to structured numerical or categorical representations generated from body information and intake history information, which are suitable for algorithmic processing, including input to a generative artificial intelligence model.

The term “description text” refers to a human-readable or machine-readable textual representation that includes the feature data and the intake history information and that summarizes the user's health context in natural language or semi-structured language.

The term “prompt sentence” refers to a textual input sequence provided to a generative artificial intelligence model, which includes the description text and instructions specifying a desired output format, such as evaluation results, recommendations, or lists of nutritional supplements.

The term “generative artificial intelligence model” refers to a machine learning model that, in response to a prompt sentence, generates output data such as natural-language text or structured text, based on parameters learned from training data, and that can perform tasks including evaluation, recommendation, and explanation.

The term “response text” refers to text data generated by the generative artificial intelligence model in response to the prompt sentence, including at least an evaluation result of potential health risks and information about recommended and not recommended nutritional supplements.

The term “potential health risk” refers to a possible adverse effect on the user's health that may arise, especially in a long-term perspective, from nutritional supplement intake or from interactions between supplement intake and the user's health status.

The term “nutritional supplements” refers to ingestible compositions containing nutrients, such as vitamins, minerals, amino acids, or other nutritional components, that are intended to support maintenance or improvement of health.

The term “identification information of the nutritional supplements” refers to data that distinguishes one nutritional supplement from another, including, for example, a name, an ingredient designation, or a code assigned to a supplement.

The term “classification information of the nutritional supplements” refers to data indicating a category or type to which a nutritional supplement belongs, such as a nutrient category, functional group, or risk category.

The term “recommended intake amounts” refers to quantitative values, such as daily dosage ranges or maximum thresholds, proposed for the intake of nutritional supplements for the user based on the evaluation result.

The term “caution information” refers to text or structured data describing precautions, warnings, contraindications, or conditions under which the intake of a nutritional supplement should be reduced, avoided, or monitored.

The term “structured data” refers to data arranged according to a predefined schema, such as records, lists, or key-value pairs, so that each element of information from the response text can be programmatically accessed and processed.

The term “rule processing” refers to an operation in which the processor applies predetermined logical rules, conditions, or constraints to the structured data in order to modify, filter, or validate the contents derived from the generative artificial intelligence model.

The term “reference information” refers to information stored in advance, such as thresholds, medical guidelines, constraint rules, or knowledge bases, that is used as criteria when executing rule processing on the structured data.

The term “final recommended nutritional supplements for intake” refers to a set of nutritional supplements, with associated conditions and amounts, that the system outputs as suitable for the user after applying rule processing to the response text of the generative artificial intelligence model.

The term “final non-recommended nutritional supplements for intake” refers to a set of nutritional supplements that the system outputs as unsuitable or to be avoided for the user after applying rule processing to the response text of the generative artificial intelligence model.

The term “warning information” refers to information that alerts the user to potential health risks, inappropriate supplement usage, or interactions, based on the evaluation result and the refined structured data.

The term “explanation information” refers to information that describes reasons, mechanisms, or contextual factors underlying the evaluation result, the recommendations, or the warnings, in a form interpretable by the user.

The term “biometric information” refers to time-series data or discrete data representing physiological measurements of the user, such as heart rate, blood pressure, body temperature, or similar indicators measured continuously or periodically.

The term “time-series data” refers to data that includes values of one or more variables recorded at multiple time points, allowing analysis of temporal changes or trends in biometric information, body information, or intake history information.

The term “emotional state information” refers to data indicating a psychological or affective condition of the user, such as stress level, mood category, or self-reported emotional state, captured through questionnaires, sensors, or other input methods.

The term “recommended intake contents” refers to a set of recommendations that specify which nutritional supplements the user should take, in what amounts, and under what conditions, based on evaluation by the system.

In one embodiment, a server cooperates with a terminal operated by a user to implement the claimed system. The server includes at least one processor, a main memory, nonvolatile storage, and a network interface connected to a communication network. The terminal includes a processor, a display, an input interface, a local memory, and a network interface. The server and the terminal execute software modules that realize acquisition of health-related data, generation of feature data, construction of a prompt sentence for a generative AI model, execution of model inference, parsing and rule-based refinement of the model output, and generation and display of warnings and explanations.

The server uses a general-purpose computing platform, such as a rack-mounted or virtualized computer system including a central processing unit (for example, a multi-core CPU) and optionally a graphics processing unit or dedicated accelerator. The server runs an operating system, such as a general-purpose server operating system, and executes an application stack including a web server component, an application framework (for example, a framework for a scripting language or an object-oriented language), a database management system, and a machine learning runtime such as a tensor computation framework. The server stores the generative AI model parameters in nonvolatile storage and loads them into memory for inference.

The terminal uses a mobile or stationary computing platform, such as a smartphone, tablet, or personal computer, running a client operating system. The terminal executes a native application or a browser-based client implemented using a user-interface framework. The terminal displays graphical input elements, receives text and numeric input from the user, and invokes network libraries to transmit and receive information to and from the server using a secure transport protocol such as HTTPS with TLS.

The user provides body information and intake history information to the terminal using input fields displayed on a graphical user interface. The user may enter values such as age, sex, height, weight, blood pressure, blood lipid values, blood glucose values, and other health-related indicators. The user may also select, from a predefined list or by free-text input, nutritional supplements being taken or previously taken, including name, approximate dose, and frequency. The user may additionally input emotional state information, for example by selecting from mood categories or entering free-text comments describing stress level or sleep quality.

The terminal converts the user's input into internal data structures, for example key-value mappings in memory, and performs preliminary validation. The terminal checks, by executing conditional evaluation code, whether required fields are present and whether numeric values fall within plausible physical ranges. The terminal then serializes the data structures into a structured format in memory and invokes a network library to encapsulate the structured data within an encrypted communication packet according to a transport-layer security protocol.

The terminal thereby transmits the body information, intake history information, biometric information (if available), and emotional state information to the server.

The server receives encrypted packets via the network interface and passes the packets through a transport-layer security stack that executes decryption and integrity verification.

The server then passes the decrypted application-layer data to an application framework, which parses the structured representation into in-memory objects. The server stores at least a subset of the raw body information and intake history information in a relational database management system, by executing insertion commands, so that the information is persistently associated with the user.

The server executes a normalization module that converts raw body information into standardized feature values. The server performs unit conversion where necessary, for example converting laboratory values from one unit system to another by applying stored conversion factors, and performs numeric scaling using predetermined or learned scaling functions. The server calculates index values such as body mass index or ratios between specific laboratory values by executing arithmetic operations over the normalized values. The server aggregates the normalized values and index values into feature data, which may be represented as a fixed-length vector of real numbers in memory. The server thereby transforms heterogeneous health data into a representation directly suitable for algorithmic processing.

The server further prepares an extended feature representation that incorporates intake history information, biometric time-series data, and emotional state information. The server encodes intake history information by mapping each supplement name or ingredient to an identifier, representing dose and frequency as numeric values, and constructing a sparse or dense vector that indicates, for each supplement category, recent and long-term exposure. The server encodes biometric information, such as periodic heart rate or blood pressure measurements, by computing summary statistics over time windows (for example, averages, variability measures, or trend coefficients) and appending these values to the feature vector. The server encodes emotional state information by mapping each state category or recognized sentiment to a numeric embedding or one-hot representation and integrating it into the feature vector. In this way, the server converts multimodal data streams into a unified feature space.

The server generates a description text from the feature data and intake history information.

The server executes string construction logic that inserts feature values and derived indices into a natural-language template, thereby producing a text describing the user's current health profile and supplement usage in a consistent, machine-generated form. For example, the server may generate a description such as:

“User is a 52-year-old male with BMI 28.0, fasting blood glucose 115 mg/dL, LDL cholesterol 160 mg/dL, HDL cholesterol 40 mg/dL, and blood pressure around 145/95 mmHg.

The user currently takes a daily multivitamin and vitamin C, and has taken omega-3 fatty acids intermittently over the last year.”

The server constructs a prompt sentence for the generative AI model by combining the description text with explicit instructions regarding desired outputs, formatting, and constraints. The prompt sentence may take a form such as:

“User profile: age 52, male, BMI 28.0, fasting blood glucose 115 mg/dL, LDL cholesterol 160 mg/dL, HDL cholesterol 40 mg/dL, blood pressure 145/95 mmHg. Current supplements: multivitamin (1 tablet/day), vitamin C 500 mg/day, intermittent omega-3. Based on this profile, identify which nutritional supplements should be recommended and which should be avoided to reduce cardiovascular and metabolic risk. List each supplement with category, recommended intake amount, or reason for avoidance, and briefly explain the potential long-term health risks.”

In another example, the server constructs a prompt sentence that explicitly incorporates biometric trends:

“Over the past 3 months, resting heart rate increased from 70 bpm to 85 bpm, and blood pressure has averaged 145/95 mmHg. Recent blood test results indicate high LDL cholesterol and borderline high fasting glucose. The user reports elevated stress levels. Considering these time-series trends and the current supplement intake, recommend nutritional supplements to support cardiovascular and metabolic health and identify any supplements or ingredients that should be avoided in the long term. Provide specific reasoning for each recommendation and avoidance.”

The server inputs the prompt sentence into a generative AI model executed locally or accessed via an API. In one embodiment, the server implements the generative AI model as a transformer-based neural network comprising an embedding layer, multiple self-attention layers, feedforward layers, and a final projection layer. The server stores model parameters, including weight matrices and bias vectors, in memory and performs inference using a tensor computation library on a CPU or accelerator. The server tokenizes the prompt sentence, maps tokens to vector embeddings, and propagates these embeddings through the transformer layers, computing attention scores and nonlinear transformations according to learned parameters. The server decodes the output log its into a sequence of tokens representing the response text.

In another embodiment, the server invokes an external model service through a network API, in which case the server transmits the prompt sentence as a text payload and receives the response text as a returned text payload. In both configurations, the server controls model parameters used at inference time, such as temperature or maximum output length, to ensure deterministic or semi-deterministic behavior suitable for medical-context explanation and recommendation.

The server trains or fine-tunes the generative AI model on a training dataset that includes pairs of prompt sentences and target responses. The server uses a loss function, such as cross-entropy over token predictions, and applies gradient-based optimization to update the model parameters. During training, the server may employ data augmentation techniques, such as perturbing numeric values within physiologically plausible ranges or rephrasing textual segments, to improve robustness. The server uses a learning rate schedule and regularization techniques to prevent overfitting. In this way, the server configures the generative AI model to reliably map structured health-context prompts to recommendation-oriented responses.

The server receives the response text generated by the generative AI model. The response text includes recommendations for nutritional supplements, identified supplements that should be avoided, and explanations of potential long-term health risks. The server executes a parsing module that transforms the response text into structured data. The parsing module applies pattern-matching rules, regular expressions, or prompt-induced delimitation (for example, requiring the model to output in a delimited list format) to extract supplement names, categories, suggested intake amounts, and caution notes. The server stores the extracted elements in a structured representation, such as a list of records in memory.

The server applies rule processing to the structured data by referencing pre-stored reference information, such as threshold tables, safety limits, or medical guideline-based rules. For example, the server may store, in a database or configuration file, maximum daily intake limits for specific nutrients, contraindication rules linking certain lab value ranges to forbidden supplements, and interaction rules concerning combinations of supplements. The server compares the extracted recommended intake amounts to these limits and adjusts or removes recommendations that conflict with the stored rules. The server may also insert additional caution notes when the generative AI model output omits identified risks that are encoded in the rule set. This non-conventional combination of model-generated outputs with deterministic, processor-enforced rules improves consistency and safety compared to purely manual review or naive model usage.

The server generates final recommended nutritional supplements for intake and final non-recommended nutritional supplements for intake, by combining the structured data with the results of rule processing. The server further generates warning information summarizing potential risks, such as long-term organ burden or cardiovascular risk associated with specific intake patterns, and generates explanation information that describes why a supplement is recommended or discouraged. The server constructs these outputs in both structured form (for internal use and logging) and natural-language form (for user display).

The server transmits the final outputs to the terminal using an encrypted communication scheme. The terminal receives the outputs, performs decryption, and parses the structured representation. The terminal displays the recommendations and warnings as separate sections on the graphical user interface, for example as “Recommended Supplements,” “Supplements to Avoid,” and “Risk Explanations.” The terminal may additionally allow the user to select an item and request more detailed explanation, in which case the terminal transmits a follow-up query to the server.

The server, upon receiving a follow-up query, constructs a secondary prompt sentence that includes the previous recommendation context and the user's specific question. The server may generate a prompt such as:

“The previous recommendation advised avoiding high-dose vitamin E due to cardiovascular risk. The user asks: ‘Why should I avoid high-dose vitamin E?’ Explain in detail, referencing the user's high LDL cholesterol and elevated blood pressure, and limit the explanation to clinically relevant mechanisms without suggesting changes to prescribed medications.”

The server inputs this secondary prompt into the generative AI model and obtains an explanation-focused response. The server optionally subjects this response to a lightweight rule check for prohibited content and then returns the explanation to the terminal. The terminal displays the explanation as an expanded detail view.

The server is configured to integrate continuous biometric information into its processing pipeline. For example, the terminal may periodically collect heart rate, blood pressure, or activity levels from connected sensing devices via an application programming interface, then transmit summarized time-series values to the server. The server updates feature data to include current and historical biometric trends and regenerates prompt sentences that explicitly describe these trends. This design enables the generative AI model to account for long-term physiological dynamics rather than static snapshots.

The server is also configured to incorporate emotional state information. The terminal may present simple questions such as “How stressed do you feel today?” with selectable options.

The terminal sends the emotional state information to the server, which encodes it and embeds it into the feature data and description text. The generative AI model thereby receives a holistic context that includes psychological conditions, allowing the system to adjust recommendations in a manner that may, for example, emphasize supplements associated with stress support while avoiding ingredients that could exacerbate anxiety or sleep issues.

The described architecture improves computer technology by providing a structured, repeatable data flow that leverages the specific strengths of both generative models and deterministic rule engines. By normalizing and encoding heterogeneous health and behavioral data into feature data before constructing the prompt sentence, the server reduces redundancy and irregularity in the input, resulting in fewer tokens and more compact, information-dense prompts. This contributes to reduced inference time and lower communication bandwidth when external models are used. Moreover, by enforcing a strict output schema via parsing and rule processing, the server enables subsequent computations—such as longitudinal risk scoring, database indexing, and caching—to operate on structured representations, thereby improving overall computational efficiency and data management.

The system's use of a generative AI model is not limited to automating a human expert's narrative, but instead cooperates with a custom data encoding and rule-application pipeline that cannot be practically executed manually. The feature generation, prompt construction, structured output extraction, and rule refinement steps are executed at machine speed over large numbers of parameters and thresholds, making it possible to adapt recommendations in near real time as new biometric and emotional data arrive. This pipeline reduces computational errors that would result from ad hoc text processing and improves accuracy and stability of the generated recommendations by constraining the model output using medically grounded rule sets.

In alternative embodiments, the server may employ different neural network architectures, such as encoder-decoder transformers, recurrent neural networks with attention mechanisms, or hybrid models combining sequence encoders with gradient-boosted decision trees for certain sub-tasks. The server may use alternative training regimes, including supervised fine-tuning on domain-specific corpora, reinforcement learning from expert feedback, or multi-task learning where both recommendation and explanation tasks share model parameters. The server may adjust loss functions to penalize incorrect risk categorization more heavily than minor textual errors, thereby directing learning toward safety-critical performance.

In another variation, the server may modify the structure of the prompt sentence to include explicit labels or delimiters that simplify downstream parsing. For example, the server may instruct the model to output its recommendations under headings such as “RECOMMENDED_SUPPLEMENTS” and “NON_RECOMMENDED_SUPPLEMENTS,” each followed by line-separated entries with predefined fields. This non-standard use of structured prompts enhances the reliability of machine parsing and thereby reduces the complexity and run time of the parser, constituting an improvement in internal computer processing.

In a further embodiment, the server may maintain separate models or separate parameter sets for different population segments, such as age groups or specific comorbidity profiles. The server dynamically selects an appropriate model or parameter configuration based on the user's profile by executing simple classification logic. This model selection at the server level improves inference accuracy and reduces the need for a single, overgeneralized model, which would require more computational resources.

The terminal and the server may be configured to cache recent feature data, prompt sentences, and model outputs in memory or local storage to accelerate repeated or similar queries. For example, when a user updates only one laboratory value, the server may reuse parts of previous feature data and prompt structures, modifying only the changed segments. This reduces processing time and conserves bandwidth, providing a tangible technical advantage over systems that reconstruct entire request payloads from scratch.

Through these configurations, the server, the terminal, and the user cooperate to implement the claimed system in a manner that not only automates certain expert-like tasks but also introduces new internal data structures, processing flows, and hybrid model-rule algorithms that achieve improved computational efficiency, accuracy, and technical robustness in health-related risk evaluation and nutritional supplement recommendation.

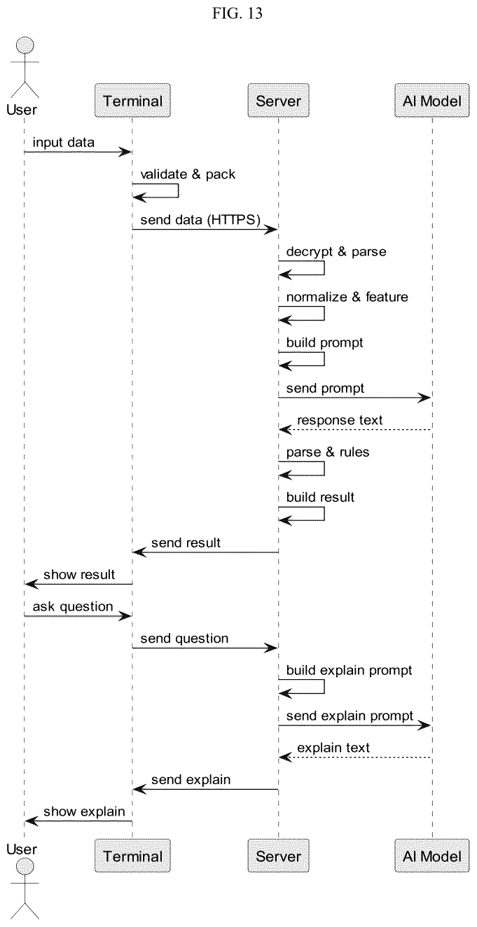

The following describes the processing flow using FIG. 13.

### Step 1:

User inputs health and supplement information into the terminal.

User enters body information such as age, sex, height, weight, blood pressure, blood lipid values, and blood glucose values into input fields shown on the terminal display, and selects or types nutritional supplements and intake amounts as intake history information. The input of this step is raw human-entered text and numeric values; the output of this step is a set of in-memory data fields on the terminal representing body information, intake history information, optional biometric information, and emotional state information.

### Step 2:

Terminal validates and structures the input data.

Terminal checks that required fields are present and within plausible numeric ranges, converts text and number inputs into internal key-value pairs, and associates each input with a data type and a unit. The input of this step is the unvalidated in-memory data fields from Step 1; the output is a validated and structured data object, for example a logical record containing typed fields such as age, sex, height, weight, laboratory values, supplement names, and doses.

### Step 3:

Terminal encrypts the structured data and transmits it to the server.

Terminal uses a secure communication library to serialize the structured data into a request payload, applies an encryption protocol such as TLS to the payload, and sends the encrypted payload to the server via a network interface. The input of this step is the structured data object from Step 2; the output is an encrypted network packet containing the user's health-related data addressed to the server.

### Step 4:

Server receives and decrypts the transmitted data.

Server accepts incoming packets on a network interface, passes them through a transport-layer security module to perform decryption and integrity verification, and parses the decrypted payload into an internal structured representation compatible with the server's application framework. The input of this step is the encrypted network packet from Step 3; the output is a server-side structured data object containing body information, intake history information, biometric information, and emotional state information for the user.

### Step 5:

Server normalizes body information and computes index values.

Server reads numeric fields such as height, weight, blood pressure, and laboratory results from the structured data, converts units where needed, scales values into normalized ranges using stored scaling factors, and computes derived indices such as body mass index and selected ratios between laboratory parameters. The input of this step is the raw server-side structured data object from Step 4; the output is a normalized feature subset including standardized body information and calculated index values.

### Step 6:

Server encodes intake history, biometric time-series, and emotional state into feature data.

Server maps supplement names to internal identifiers, converts doses and frequencies into numeric features, aggregates biometric measurements into summary statistics (for example, average heart rate or blood pressure trends), and encodes emotional state categories into numeric representations. The input of this step is the structured intake history information, biometric information, and emotional state information from Step 4; the output is a combined feature data vector that integrates body information, index values, intake history, biometric summaries, and emotional state into a unified numerical representation.

### Step 7:

Server generates a description text from the feature data.

Server takes the combined feature data vector and inserts feature values into predetermined natural-language templates, assembling sentences that describe the user's health status, supplement usage, biometric trends, and emotional state. The input of this step is the unified feature data generated in Steps 5 and 6; the output is a description text that summarizes the user's health context in machine-generated natural language.

### Step 8:

Server constructs a prompt sentence for the generative AI model.

Server combines the description text with instruction segments that specify the required output format, such as listing recommended and non-recommended supplements, including categories, dosages, and reasons. The input of this step is the description text from Step 7; the output is a prompt sentence string designed for input to the generative AI model.

### Step 9:

Server executes inference of the generative AI model using the prompt sentence.

Server tokenizes the prompt sentence, converts tokens into embeddings, propagates the embeddings through a generative neural network (for example, a transformer with multiple attention layers), and decodes output token probabilities into response text. The input of this step is the prompt sentence from Step 8; the output is a response text that includes an evaluation of potential health risks, a list of recommended nutritional supplements, and a list of supplements to avoid for the user.

### Step 10:

Server parses the response text into structured data.

Server applies parsing logic, such as pattern matching or delimiter-based splitting, to the response text to identify supplement names, classification information, recommended intake amounts, and caution information, and stores each extracted element into defined fields of a structured representation. The input of this step is the free-form response text from Step 9; the output is a structured data object that organizes supplements, categories, dosages, and warnings into machine-readable records.

### Step 11:

Server applies rule processing to refine recommendations.

Server compares the structured data object to reference information containing thresholds, safety limits, and contraindication rules, and modifies, removes, or augments entries where the generative AI output conflicts with these rules. The input of this step is the structured recommendation data from Step 10 and the stored reference rule set; the output is corrected and validated recommendation data that encodes final recommended supplements and final non-recommended supplements in compliance with the reference information.

### Step 12:

Server generates warning information and explanation information.

Server uses the refined recommendation data and the health risk evaluations to construct warning messages and explanatory texts, combining template-based phrasing with context derived from the user's feature data. The input of this step is the corrected recommendation data from Step 11; the output is a pair of outputs: warning information specifying potential risks and explanation information describing the reasoning behind each recommendation or avoidance.

### Step 13:

Server transmits final recommendations, warnings, and explanations to the terminal.

Server serializes the refined structured data and associated texts into a response payload, encrypts the payload using the established communication protocol, and sends the encrypted payload back to the terminal over the network. The input of this step is the final recommendation data and explanation data from Steps 11 and 12; the output is an encrypted response packet addressed to the terminal.

### Step 14:

Terminal receives, decrypts, and parses the server response.

Terminal accepts incoming encrypted packets, performs decryption using its security stack, and converts the decrypted payload into internal objects representing recommended supplements, supplements to avoid, and associated warnings and explanations. The input of this step is the encrypted response packet from Step 13; the output is a terminal-side structured data object ready for display.

### Step 15:

Terminal displays recommendations and warnings to the user.

Terminal renders user interface elements that list recommended supplements, non-recommended supplements, and risk explanations, grouping them visually and associating each item with its explanation, and updates the screen to show this information. The input of this step is the terminal-side structured data object from Step 14; the output is a visual representation on the display that the user can read and interact with.

### Step 16:

User reviews the displayed information and optionally submits a follow-up question.

User inspects the recommended and non-recommended supplements and may type or select a question such as a request for more details about a specific item, and confirms the question through the terminal interface. The input of this step is the displayed information from Step 15; the output is new user-generated query text and a selection context (for example, a particular supplement) held in terminal memory.

### Step 17:

Terminal sends the follow-up question and context to the server.

Terminal packages the user's question together with identifiers of the relevant recommendation items into a structured object, encrypts the object using the same secure communication mechanism, and transmits it to the server. The input of this step is the follow-up query text and context from Step 16; the output is an encrypted follow-up request packet destined for the server.

### Step 18:

Server constructs a context-aware prompt sentence for explanation and executes model inference.

Server merges the previously stored feature data, the prior recommendation context, and the user's follow-up question to construct a new prompt sentence that instructs the generative AI model to explain, without changing the recommendation, the reasons for a certain warning or suggestion. The input of this step is the follow-up request from Step 17 and stored context from previous steps; the output is a secondary prompt sentence and a subsequent explanation response text produced by another inference pass of the generative AI model.

### Step 19:

Server post-processes the explanation response and returns it to the terminal.

Server optionally checks the explanation response against simple safety filters, packages the explanation text into a response payload, encrypts it, and sends it back to the terminal. The input of this step is the explanation response text from Step 18; the output is an encrypted explanation packet transmitted to the terminal.

### Step 20:

Terminal displays the detailed explanation to the user.

Terminal decrypts the received explanation packet, extracts the explanation text, and displays the text in association with the relevant supplement or warning, for example in an expanded view or detail screen. The input of this step is the decrypted explanation payload from Step 19; the output is a visual explanation on the terminal display that the user can read to understand the technical basis of the recommendation.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented systems that provide recommendations for health support intake targets, such as nutritional supplements or similar consumables, suffer from several technical limitations in how they acquire, structure, process, and utilize heterogeneous user data.

First, existing systems typically apply fixed rule sets or simple scoring logic to static user inputs. These systems do not dynamically aggregate and preprocess diverse time-series data, including usage history information, biometric measurement information, emotion state information, and purchase history information, in a unified computational pipeline. As a result, the systems cannot fully exploit temporal correlations between intake events and subsequent changes in biometric indicators or emotion states, leading to low information density and underutilization of available data in the internal representations. This limits the quality and reliability of downstream computation, such as risk evaluation or recommendation generation.

Second, even when machine learning components are used, known systems generally feed raw or minimally formatted data into the models. They do not systematically convert aggregated multi-modal, time-series inputs into structured natural-language explanatory text and do not construct prompt sentences that explicitly specify analysis targets and output formats for a generative AI model. Without such prompt engineering performed by the processor itself, the generative AI model cannot be consistently guided to perform context-aware risk evaluations and recommendation tasks. This leads to unstable or ambiguous outputs, and imposes additional processing burdens on the system to interpret and normalize results, degrading computational efficiency and robustness.

Third, current architectures do not integrate, within a single processor-controlled workflow, (i) automatic extraction and structuring of natural-language outputs from a generative AI model, (ii) iterative, periodic re-evaluation of long-term effects and long-term risks for each intake target based on historical time-series data, and (iii) generation of machine-readable, user-specific intake recommendation information and health impact risk evaluation information. Fragmented components, if present, often require manual intervention or ad-hoc scripting to connect model outputs to transaction flows and feedback loops, resulting in increased latency, higher computational overhead, and reduced scalability.

Fourth, many known systems handle feedback to users in a static or one-shot manner. They do not cause the processor to dynamically generate multiple types of prompt sentences for the same underlying usage history information and health state evaluation result information, with different generation purposes such as alerts, recommendations, and long-term effect summaries. Consequently, such systems are unable to obtain complementary analysis results from a generative AI model in a coordinated and automated way, and cannot refine or cross-validate model outputs across different prompts. This reduces the reliability of the system's decision-support functionality and prevents effective improvement of the internal inference process over time.

Fifth, the coupling between analytical results and downstream transaction processing is typically weak. Existing systems often treat electronic payment as an external, separate function, rather than an integrated part of the computational pipeline. They do not configure the processor to use structured recommendation outputs, including item-level and dosage-level data, as direct input for constructing electronic payment requests, executing payment processing, and feeding transaction results back into the data store for use in subsequent analyses. This separation prevents the system from using actual purchase behavior as a first-class data source in the analytic loop and obstructs optimization of end-to-end system performance.

Accordingly, there is a need for an improved computer-implemented system in which a processor itself orchestrates: (i) unified aggregation and time-series preprocessing of heterogeneous user data, (ii) automatic construction of prompt sentences tailored to a generative AI model, (iii) structured parsing of natural-language outputs, (iv) periodic re-evaluation of long-term effects and risks, and (v) integrated generation of presentation information and transaction processing. Such a system would technically improve the way computers store, represent, and transform health-related multi-modal data into reliable, machine-usable outputs, thereby enhancing computational efficiency, scalability, and stability of generative-AI-assisted recommendation and risk evaluation workflows.

The specific processing by the specific processing unit 290 of the data processing device 12 in Application Example 2 is realized by the following means.

The present invention provides a server comprising a processor configured to receive usage history information of multiple types of intake targets for health support from a terminal device, store the usage history information, health state evaluation result information, biometric measurement information, emotion state information, and purchase history information in a storage device, aggregate and preprocess the stored information on a time-series basis to form unified internal representations, convert the aggregated and preprocessed information into explanatory text expressed in natural language, construct a prompt sentence including an instruction statement that specifies an analysis target and an output format based on the explanatory text, input the prompt sentence to a generative AI model, obtain a natural-language output from the generative AI model, extract, from the natural-language output, names of intake targets for health support, recommended amounts, usage cautions, and contraindications, convert the extracted information into structured data, generate, based on the structured data, intake recommendation information and long-term health impact risk evaluation information for each user, dynamically generate different types of prompt sentences for the same underlying data in accordance with at least one generation purpose selected from alert information, intake recommendation information, and long-term effect evaluation, perform periodic re-evaluation of long-term effects and long-term risks for each intake target based on historical time-series data using automatically generated prompt sentences, generate presentation information for supporting selection and dosage adjustment of the intake targets for health support by the user based on the intake recommendation information and the long-term health impact risk evaluation information, transmit the presentation information to the terminal device via a network, receive transaction request information from the terminal device, execute an electronic payment process based on product information of the intake targets for health support included in the intake recommendation information and the transaction request information, and return transaction result information corresponding to a payment result to the terminal device. This enables the computer system to internally orchestrate data aggregation, prompt construction, generative-model interaction, structured result extraction, iterative long-term evaluation, and transaction processing as a unified, machine-implemented pipeline, thereby improving the efficiency, stability, and reliability of health-related recommendation and risk evaluation operations performed by the processor.

The term “processor” refers to a hardware computation unit or a combination of hardware computation units configured to execute instructions of a program and thereby perform arithmetic operations, logical operations, data transfer, and control of other components of a system.

The term “system” refers to a combination of one or more server devices, one or more terminal devices, storage resources, communication interfaces, and software components that cooperate via a communication network to perform the processing described in the claims.

The term “usage history information” refers to data indicating past and current use of intake targets for health support by a user, including at least an identifier of each intake target, an amount or dosage, a frequency, a time or date of intake, and an association with the user.

The term “intake target for health support” refers to a consumable product or composition intended to supplement or support a biological function of a user, such as a nutritional supplement, health-support food, or similar ingestible material.

The term “potential long-term health impact risk” refers to a likelihood or degree of adverse or beneficial influence on a user's health that may arise from continued intake of one or more intake targets for health support over an extended period.

The term “generative information processing model” refers to a trained computational model that receives input data and outputs generated content, such as natural-language text, based on learned statistical relationships, and that is capable of producing new candidate explanations, evaluations, or recommendations rather than only classifying or scoring inputs.

The term “alert information” refers to machine-generated data that indicate a caution, warning, or recommendation for attention by a user in view of a calculated potential long-term health impact risk or other evaluation.

The term “health state evaluation result information” refers to data obtained from a measurement or assessment of a user's health condition, including, for example, laboratory values, vital signs, or medically relevant indices output from a health examination.

The term “user information” refers to data associated with a particular user, including at least the user's usage history information and health state evaluation result information, and optionally including demographic or profile information.

The term “biometric measurement information” refers to time-dependent or event-dependent data that quantitatively indicate a physiological state of a user, such as heart rate, blood pressure, body temperature, or other measurable biological indicators.

The term “emotion state information” refers to data representing a psychological or affective state of a user, such as stress, joy, anxiety, or similar emotional conditions, the data being derived from analysis of user input or sensor signals.

The term “purchase history information” refers to records of previous transactions related to intake targets for health support by a user, including at least product identifiers, quantities, transaction times, and, optionally, payment results.

The term “storage device” refers to one or more physical or virtual data-storage components, such as magnetic disks, solid-state memories, or network-attached storage, used for persistently or semi-persistently storing information.

The term “aggregate and preprocess” refers to operations that collect, align, normalize, filter, and transform raw data from multiple sources into consistent, combined, and structured forms suitable for subsequent analysis or model input.

The term “time-series basis” refers to processing or organizing data according to temporal order, including associating each data element with a time stamp and analyzing sequences or trends over time.

The term “explanatory text expressed in natural language” refers to human-readable sentences or paragraphs generated or composed in a natural language, such as English, that describe or summarize underlying structured or time-series data.

The term “prompt sentence” refers to a sequence of one or more natural-language statements that is supplied as input to a generative information processing model and that instructs the model regarding a task to perform, an analysis target, and an expected output format.

The term “instruction statement that specifies an analysis target and an output format” refers to a portion of a prompt sentence that explicitly indicates what data or subject matter is to be analyzed by the generative information processing model and in what structure or style the model should return its output.

The term “natural-language output” refers to a sequence of tokens, words, or sentences in a natural language produced by the generative information processing model in response to an input prompt sentence.

The term “structured data” refers to information organized in a machine-processable format with explicit fields or keys, such as records, tables, or objects, that represent specific attributes including names of intake targets for health support, recommended amounts, cautions, or contraindications.

The term “intake recommendation information” refers to structured data that specify which intake targets for health support are suggested for a user, together with recommended doses, timings, and optional explanatory reasons.

The term “long-term health impact risk evaluation information” refers to structured data indicating an assessment of potential long-term health risks or benefits associated with the user's intake of one or more intake targets for health support.

The term “presentation information” refers to data formatted for display or user interaction on a terminal device, including text, symbols, or graphical elements that convey intake recommendations, alerts, risk evaluations, or related guidance.

The term “selection and dosage adjustment” refers to user actions of choosing which intake targets for health support to use and modifying amounts, frequencies, or schedules of intake based on presentation information.

The term “terminal device” refers to an endpoint computing device operated by or near the user, such as a mobile communication device, a wearable device, or a general-purpose client device, that communicates with the server via a network.

The term “network” refers to a communication infrastructure comprising one or more wired or wireless links and communication protocols that enable data exchange between the server and one or more terminal devices.

The term “transaction request information” refers to data sent from a terminal device to the server that indicate a user's intention to perform a commercial transaction for one or more intake targets for health support, including product identifiers, quantities, and, optionally, delivery and payment parameters.

The term “electronic payment process” refers to a series of computational operations executed by the processor to perform electronic transfer of funds or payment authorization based on transaction request information and product information.

The term “transaction result information” refers to data indicating an outcome of an electronic payment process, including success or failure indicators, error codes, and, optionally, settlement details.

The term “historical time-series data” refers to stored data that include sequences of usage history information, biometric measurement information, and emotion state information, each associated with time stamps and spanning a predetermined past period.

The term “long-term effects” refers to changes in biometric measurement information or emotion state information that are observed over an extended time period and that can be associated with continuous or repeated intake of an intake target for health support.

The term “long-term risks” refers to potentially adverse long-term effects or increased likelihood of negative health outcomes attributable to continued intake of one or more intake targets for health support.

The term “follow-up feedback information” refers to presentation information generated after an initial recommendation that reports re-evaluated long-term effects or risks and suggests whether to continue, adjust, or discontinue intake targets for health support.

The term “statistical values of biometric indicators” refers to numerical summaries derived from biometric measurement information, including at least averages, variances, trends, or other statistical metrics computed over specified time intervals.

The term “occurrence frequencies of emotion states” refers to numerical counts or ratios that indicate how often specific emotion states appear within defined time windows in the emotion state information.

The term “generation purpose” refers to a predefined functional objective for interaction with the generative information processing model, such as producing alert information, generating intake recommendation information, or summarizing long-term effects.

The term “complementary analysis results” refers to multiple outputs from the generative information processing model that are obtained using different prompt sentences based on the same underlying data and that provide additional, cross-checking, or mutually reinforcing perspectives on risk or recommendation.

In one embodiment, a server executes a computer program that implements the functions of the claimed system. The server includes at least one processor, a main memory, a non-volatile storage device, and a network interface. The processor is, for example, a general-purpose CPU or a combination of a CPU and a graphics processing unit. The non-volatile storage device stores executable program modules, configuration files, and databases. The network interface provides connectivity to one or more terminal devices operated by users.

The server stores a relational database management system, for example a database engine of the type of a structured query language database. The server stores application logic implemented by an application framework, for example a web application framework written in a high-level programming language. The server further stores machine learning libraries, for example numerical computation libraries and a deep learning framework, and a natural language processing library. The server communicates with a generative AI model via an application programming interface exposed by a remote inference service or by a local model-serving component.

The terminal is a user-operated computational device such as a smartphone, a tablet, or a wearable device. The terminal includes a processor, a display, an input interface, and communication circuitry. The terminal executes a user interface program, for example a mobile application or a browser-based application implemented in a client-side framework.

The terminal communicates with the server over a communication network by using secure transport protocols. The terminal may also communicate with a wearable sensing device through a short-range wireless protocol.

The user operates the terminal to enter health state evaluation result information, such as laboratory values, blood pressure values, or diagnostic labels, and to input usage history information of intake targets for health support. The user may, for example, select a product name from a catalog, enter a dose, and specify a time of intake. The user may additionally provide emotion state information by entering free-form text describing feelings or stress, or by speaking into a microphone of the terminal. The user may confirm purchase requests of recommended intake targets by operating the user interface presented on the display of the terminal.

The server receives usage history information, health state evaluation result information, biometric measurement information, emotion state information, and purchase history information from the terminal. The server stores these data in separate but related tables in the database. For example, the server stores usage history information in a first table having fields such as user identifier, intake target identifier, dose, unit, intake timestamp, and source device.

The server stores health state evaluation result information in a second table having fields such as user identifier, test type, measured value, unit, reference range, and measurement timestamp. The server stores biometric measurement information, such as heart rate or blood pressure measurements received from the terminal or a wearable device, in a third table indexed by time. The server stores emotion state information in a fourth table which includes a categorical emotion label and an associated probability score for each time interval. The server stores purchase history information, such as completed and failed transactions for intake targets for health support, in a fifth table with transaction timestamps and identifiers.

The server uses a data aggregation module implemented in a language such as a high-level scripting language and numerical libraries to aggregate and preprocess the stored information on a time-series basis. The server performs operations such as aligning time stamps from different data sources to a common time grid, resampling biometric measurement information to fixed intervals, interpolating missing values, and joining biometric measurement information with usage history information by using window functions defined in the database or in the numerical libraries. By performing such alignment and joining operations, the server associates sets of biometric measurement information and emotion state information with windows before and after intake events of particular intake targets for health support.

The server computes summary statistics for each user and for each intake target. The server calculates, for example, averages, standard deviations, and trends for biometric indicators in predetermined windows before initiation of a given intake target and in windows after initiation. The server calculates frequencies of occurrence of particular emotion states, such as stress or joy, in association with periods in which the user is taking a particular intake target.

The server stores these summary statistics as records in an internal feature table. The server in this manner transforms large volumes of heterogeneous raw data into compact, structured representations that are suitable as high-level features for downstream processing.

The server converts the aggregated and preprocessed information into explanatory text expressed in natural language. The server uses templates and rule-based formatting logic to generate descriptive sentences that summarize the user's current health state, usage history of intake targets, biometric trends, and emotion patterns. For example, the server generates an explanatory text such as:

“User A has taken a vitamin D supplement at 1000 international units each morning for the last 8 weeks. During this period, the average resting heart rate decreased from 85 beats per minute to 78 beats per minute, and systolic blood pressure remained stable around 120 mmHg. Stress-related emotion states decreased in frequency by 25 percent compared with the previous 8 weeks.”

The server then constructs a prompt sentence based on this explanatory text. The server appends an instruction statement to specify an analysis target and an output format. For example, the server constructs a prompt sentence such as:

### “User's Health Check Results:

Vitamin D deficiency (25(OH)D=18 ng/mL)
LDL cholesterol=150 mg/dL

### Current Supplements:

Vitamin D 1000 IU each morning at 8:00
Omega-3 1000 mg each evening at 20:00

### Biometric Summary (Last 4 Weeks):

Average resting heart rate decreased from 85 bpm to 78 bpm after starting Vitamin D.
Blood pressure is stable around 120/80 mmHg.

### Emotion Summary:

Self-reported stress episodes decreased by 25% after starting magnesium.

### Task:

As a medical supplement advisor, evaluate potential long-term health risks of the current supplement regimen. Propose which intake targets for health support should be continued, which should be adjusted or avoided, and explain the reasoning in simple terms.”

The server uses such a prompt sentence as input to a generative AI model. In one implementation, the generative AI model is a transformer-based neural network having multiple encoder-decoder layers, multi-head self-attention mechanisms, and layer normalization. The generative AI model has been trained on a large corpus of natural-language documents and fine-tuned on domain-specific data describing relationships between intake targets for health support and health states. During training, the model parameters are updated by using a gradient-based optimization algorithm such as stochastic gradient descent with adaptive moment estimation. The model minimizes an objective function such as a cross-entropy loss between predicted tokens and reference tokens. The server or a separate training system performs data preprocessing for training by tokenizing input texts into subword units, adding positional encodings, and performing data augmentation such as synonym replacement or paraphrasing.

The server interacts with the generative AI model through an inference interface. The server passes the prompt sentence as a sequence of tokens into the model, together with inference parameters such as a maximum output length and a sampling temperature. The generative AI model computes internal representations of the prompt sentence in multiple layers, applying attention weights that capture relationships between parts of the input, and outputs a sequence of tokens that represent a natural-language output. The server receives the output as text.

The server parses the natural-language output by using a combination of pattern-matching rules and a natural language processing library. The server extracts names of intake targets for health support, recommended amounts, usage cautions, and contraindications. For instance, the server identifies phrases such as “recommend vitamin D at 2000 IU per day” or “avoid high-dose vitamin A” and maps them to structured fields. The server normalizes units and dosage ranges to internal canonical forms. The server detects negative or cautionary expressions and associates them with corresponding intake targets. The server compiles the extracted information into structured data records. The server stores these records as intake recommendation information and long-term health impact risk evaluation information for each user.

The server generates presentation information for supporting selection and dosage adjustment of intake targets by the user. The server converts structured data into a format suitable for rendering on the terminal, such as a JSON representation of recommended items, suggested doses, reasons, and warning messages. The server also generates textual summaries for display. For example, the server produces a message such as:

“Recommended: Vitamin D 1000 to 2000 IU per day, continue current dose. Caution: consider monitoring calcium levels. Avoid: high-dose vitamin A due to potential toxicity with long-term use.”

The server transmits the presentation information to the terminal via the network. The terminal receives the presentation information and renders it as user interface elements, such as lists, color-coded labels, or graphical charts.

When the user selects one or more recommended intake targets for purchase, the terminal transmits transaction request information to the server. The server uses the intake recommendation information, including item identifiers and recommended quantities, to construct an electronic payment request. The server interfaces with an electronic payment service by transmitting parameters such as total amount, currency, and product identifiers. The server receives a payment result and returns transaction result information to the terminal. The server also records the transaction in the purchase history information. Because the server uses structured recommendation results directly as input for its payment module, the server reduces intermediate conversions and improves end-to-end throughput.

The server periodically re-evaluates long-term effects and long-term risks for each intake target. The server retrieves historical time-series data over predetermined periods, such as three months or six months, and recomputes summary statistics of biometric measurement information and emotion state information relative to intake events of each intake target. The server then constructs new prompt sentences that explicitly ask for evaluation of long-term trends. For example, the server may generate a prompt sentence such as:

“The user has taken Vitamin D 1000 IU daily for 12 weeks. Biometric trends: resting heart rate decreased from 85 bpm to 78 bpm and remained stable; blood pressure remained in the range 118-124/76-82 mmHg. Emotion trends: reported stress episodes decreased by 30% after week 4 and remained lower. Task: evaluate the long-term effect of Vitamin D supplementation on cardiovascular stability and stress reduction for this user, and suggest whether to continue, adjust, or stop this intake target for health support.”

The server submits this prompt sentence to the generative AI model, receives an updated natural-language output, and converts it again into structured data. The server forms follow-up feedback information by comparing current recommendations and risk evaluations with previous ones, and signals changes, such as “continue current dose” or “consider tapering.”

The server transmits the follow-up feedback information to the terminal, which displays updated guidance to the user.

The server is further configured to dynamically vary prompt sentences for the same underlying data in accordance with generation purposes. The server may generate, from a single set of usage history information and health state evaluation result information, one prompt sentence directed to generating alert information, another prompt sentence directed to generating intake recommendation information, and a third prompt sentence directed to summarizing long-term effects. By sending multiple, differently framed prompt sentences to the generative AI model, the server obtains complementary analysis results. The server compares and cross-checks these results internally, thereby detecting inconsistent or low-confidence outputs and reducing the risk of erroneous recommendations. This multi-prompt strategy is not a mere automation of human reading and writing but an algorithmic technique that leverages the model's generative capabilities in distinct modes and combines outputs for internal consistency checking.

The terminal may, in another embodiment, perform preliminary emotion analysis locally. The terminal may execute a sentiment analysis model implemented in a compact neural network or use an external sentiment analysis service. The terminal analyzes user text or voice transcripts to produce emotion state information represented as a categorical label and confidence score.

The terminal sends the emotion state information, rather than raw text or voice features, to the server. This reduces network bandwidth usage and improves privacy, while still providing the server with machine-readable emotion data. The server uses the emotion state information as additional features in its statistical and neural analyses of intake effects.

The system provides technical improvements over conventional techniques by defining specific data structures, computational pipelines, and model-interaction procedures. Because the server aggregates heterogeneous time-series data and computes derived statistical features before constructing prompt sentences, the server reduces the dimensionality and noise in the data that the generative AI model receives. This preprocessing reduces inference time and improves stability of the model's responses. Additionally, prompt sentences that explicitly define analysis targets and output formats reduce ambiguity and lower the number of tokens needed in the model's response, which in turn reduces computational load and latency. The extraction of structured data from natural-language outputs allows the server to cache and reuse model-derived knowledge efficiently, avoiding redundant calls to the generative AI model for repeated queries.

The coordinated design of relational schemas, feature computation, prompt construction, model interaction, and structured output parsing constitutes a specific improvement in computer technology relating to data management and machine-assisted inference. The server does not simply automate human professional judgment; instead, the server transforms raw multi-modal streams into optimized internal representations, orchestrates generative model calls with non-conventional prompt engineering based on computed features, and integrates outputs into subsequent technical processes such as risk scoring, anomaly detection, and resource-efficient transaction processing. These mechanisms yield measurable technical benefits, such as reduced inference time per user profile, increased accuracy of risk classification compared with rule-based baselines, improved data compression in the feature tables, and reduced network traffic by transmitting compact features and structured results instead of raw sensor streams and complete narratives.

In alternative embodiments, the server may deploy the generative AI model on a dedicated inference server within the same data center instead of using an external service. In such embodiments, the server may manage a model-serving component that exposes a model inference API over an internal network. The generative AI model may differ in architecture, for example being a recurrent neural network or a hybrid convolutional-transformer model, but still receives prompt sentences and outputs natural-language text. In another variation, the server may employ a combination of a generative AI model and a discriminative classifier.

The server may use the generative AI model for generating explanations and the classifier for generating numeric risk scores. The server may also vary the feature sets according to available sensors, for example using additional cardiological indicators or sleep metrics if provided by the wearable device.

The terminal may differ in form factor, for example being a smart watch that directly displays simplified recommendations without full textual explanations. In that case, the server may send more compact presentation information, such as binary flags indicating “safe,” “caution,” or “avoid” status for each intake target, along with numeric risk scores. In another variation, the terminal may be a desktop computer running a web browser, and the user interface may include advanced visualization components such as time-series plots of biometric indicators overlaid with intake timelines.

The user may use the system not only for nutritional supplements but for other intake targets for health support, provided that the data schemas and models are configured appropriately.

The server can adapt to such uses by modifying mapping tables between intake target names and internal identifiers, updating training data for the generative AI model, and adjusting rule-based safety thresholds. The overall pipeline of data aggregation, feature computation, prompt construction, generative inference, structured extraction, and feedback generation remains the same, thus providing a general technical framework for complex, time-dependent recommendation and risk evaluation tasks.

By implementing these embodiments, the system enables other persons skilled in the art to carry out the invention using known hardware and software components, while preserving the specific technical contributions in the form of the defined data structures, computational flows, prompt sentence strategies, and model-interaction mechanisms.

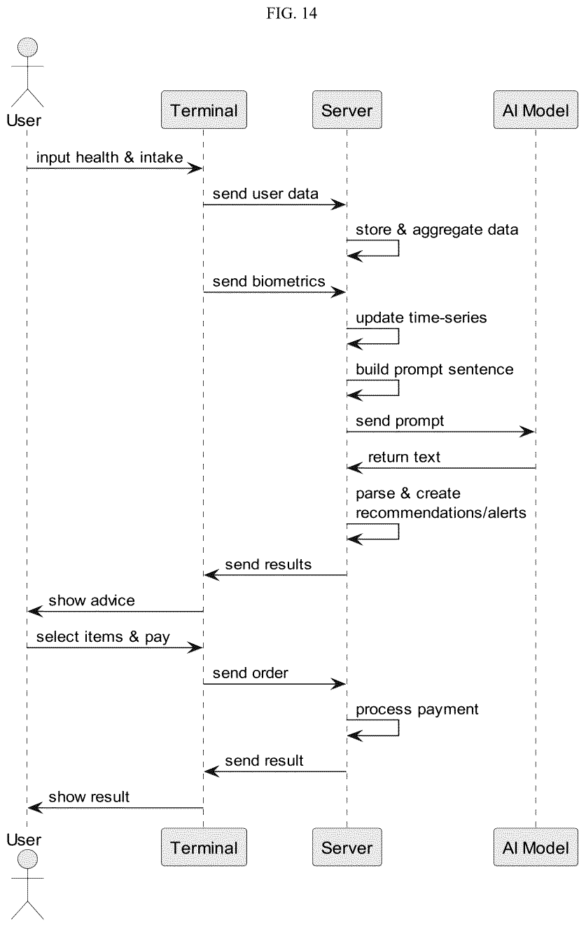

The following describes the processing flow using FIG. 14.

### Step 1:

User operates the terminal to input health and intake information.

User enters health state evaluation result information, such as numeric laboratory values, blood pressure, and diagnostic labels, into input fields of an application running on the terminal. User further inputs usage history information of intake targets for health support by selecting product names from a list, entering dosages (for example, “1000 IU”), and specifying intake times and frequencies. User may additionally type free-text about current feelings (for example, “I feel stressed lately”) or confirm previously suggested intake plans.

Input: raw user keystrokes, touch events, and optional voice input on the terminal.

Output: a structured data object inside the terminal, including fields for health state evaluation result information, usage history information, and optional free-form text, for example as an internal key-value structure.

Terminal converts the user's raw interactions into typed fields, validates numeric ranges (for example, ensuring that a cholesterol value is within a plausible range), and prepares the structured object for transmission.

### Step 2:

Terminal transmits structured user data to the server.

Terminal packages the structured data object into a message and attaches a user identifier and a timestamp. Terminal invokes a secure communication library to send the message to an API endpoint on the server over an encrypted channel. Terminal may compress the payload to reduce transmission size.

Input: structured data object constructed in Step 1 and network connection parameters.

Output: an HTTP or similar request containing serialized structured data transmitted to the server, and an internal status flag indicating successful or failed transmission.

Terminal performs basic error handling, such as retrying the request if a transient network error is detected.

### Step 3:

Server receives user data and stores it in a database.

Server listens on an API endpoint and accepts incoming requests from terminals. Server parses the received message body, verifies authentication tokens, and validates the schema of the structured data. Server maps each field to one or more database columns and executes insert operations into tables for health state evaluation result information, usage history information, and emotion-related text or labels.

Input: serialized structured data received over the network.

Output: persistent records in the database tables, each associated with a user identifier and timestamps; an acknowledgment response to the terminal indicating success or specifying validation errors.

Server may normalize vocabulary, for example mapping “Vit D” to an internal code for a specific intake target, before committing records.

### Step 4:

Terminal acquires biometric measurement information from a wearable device.

Terminal establishes a short-range wireless connection with a wearable sensor, for example a smart band that measures heart rate and blood pressure. Terminal periodically requests or subscribes to biometric measurements and receives sensor readings with timestamps.

Terminal converts vendor-specific formats to a standard internal format and tags them with a user identifier.

Input: raw sensor packets received from the wearable device.

Output: a list of biometric measurement records inside the terminal, each containing a timestamp, measurement type (for example, heart rate), and value.

Terminal may filter out obviously erroneous measurements, such as non-physiological values, before retaining the records.

### Step 5:

Terminal transmits biometric measurement information to the server.

Terminal batches biometric records collected over a configurable interval and constructs a structured message representing time-series biometric measurement information. Terminal sends this message to a biometric API endpoint on the server using an encrypted transport protocol.

Input: list of biometric measurement records stored on the terminal.

Output: a network request carrying the biometric measurement information to the server and an internal status code reflecting delivery success.

Terminal may apply simple compression or down-sampling if the volume of data is high.

### Step 6:

Server ingests biometric measurement information and updates time-series storage.

Server receives the biometric payload, validates measurement types and value ranges, and stores the records in a dedicated time-series table keyed by user identifier and timestamp.

Server may convert measurement units to standard units (for example, converting mmHg values to a normalized scale if necessary).

Input: structured biometric measurement information received from the terminal.

Output: new time-series entries in the biometric measurement table, including derived indices such as rolling averages or flags for missing intervals.

Server may trigger internal jobs when a threshold amount of new data becomes available.

### Step 7:

Server generates emotion state information from user input or receives it from the terminal.

Server may receive free-form emotion-related text directly from the terminal or a pre-computed emotion label and confidence score if the terminal performs local emotion analysis. When only text is received, server applies a natural language processing library to classify the text into discrete emotion categories and to assign probabilities.

Input: emotion-related user text or pre-computed emotion state labels.

Output: emotion state information stored in a database table, including the emotion category (for example, stress, joy), a numeric score, and timestamps.

Server uses a text-processing algorithm, such as tokenization and application of a sentiment classifier, to transform raw text into discrete labels and scores.

### Step 8:

Server aggregates multi-modal data for a target evaluation period.

Server selects a time window, for example the last four weeks, and queries the database for all relevant records: usage history information, health state evaluation result information, biometric measurement information, and emotion state information for the user. Server uses a numerical library to align records to a common timeline, join intake events with biometric measurements that occur before and after those events, and count occurrences of each emotion category within pre-intake and post-intake windows for each intake target.

Input: raw database records retrieved by time and user identifier.

Output: an internal aggregated dataset containing, for each intake target, time-aligned biometric sequences, summary statistics (for example, mean and standard deviation before and after initiation), and emotion occurrence frequencies.

Server computes these statistics by applying vectorized operations such as group-by, windowed averages, and histogram counts to arrays of timestamps and values.

### Step 9:

Server computes derived features and stores them as feature records.

Server converts the aggregated dataset into high-level features, such as change in average resting heart rate, variability of blood pressure, and percentage change in stress frequency for each intake target. Server stores these derived features in a feature table keyed by user identifier and intake target identifier.

Input: aggregated dataset from Step 8.

Output: feature records that capture time-series effects of intake targets as compact numerical descriptors.

Server performs data operations such as subtraction of pre-intake and post-intake averages, normalization by baseline values, and encoding of categorical variables into numeric form.

### Step 10:

Server generates explanatory text from feature records.

Server reads feature records and associated raw metrics and uses template rules to build natural-language descriptions. Server selects templates depending on whether metrics improved, worsened, or stayed stable. For example, if resting heart rate decreased by more than a threshold, server uses a phrase indicating improvement.

Input: feature records and metadata describing intake targets and health metrics.

Output: explanatory text in natural language summarizing usage history, biometric trends, and emotion patterns for the user.

Server concatenates sentences while enforcing grammatical correctness and inserts numeric values into placeholders in the templates.

### Step 11:

Server constructs a prompt sentence for the generative AI model.

Server takes the explanatory text from Step 10 and appends an instruction section that specifies an analysis target (for example, long-term health risk evaluation) and expected output format (for example, list of recommended intake targets and warned-against intake targets with reasons). Server organizes the information into sections, such as “Health check results,” “Current supplements,” “Biometric summary,” and “Emotion summary,” followed by a “Task” description.

Input: explanatory text, configuration parameters specifying the type of analysis requested, and any system-defined constraints.

Output: a prompt sentence expressed in natural language, formatted as a multi-paragraph string for input to the generative AI model.

Server may, for example, output a prompt sentence of the form:

### “User's Health Check Results:

Vitamin D deficiency (25(OH)D=18 ng/mL)
LDL cholesterol=150 mg/dL

### Current Supplements:

Vitamin D 1000 IU each morning at 8:00
Omega-3 1000 mg each evening at 20:00

### Biometric Summary (Last 4 Weeks):

Average resting heart rate decreased from 85 bpm to 78 bpm after starting Vitamin D.
Blood pressure is stable around 120/80 mmHg.

### Emotion Summary:

Self-reported stress episodes decreased by 25% after starting magnesium.

### Task:

As a medical supplement advisor, evaluate potential long-term health risks of the current intake regimen. Propose which intake targets for health support should be continued, which should be adjusted or avoided, and explain the reasoning in simple terms.”

### Step 12:

Server sends the prompt sentence to the generative AI model and obtains a natural-language output.

Server invokes an inference API, passing the prompt sentence as input, together with parameters such as maximum token count and sampling temperature. The generative AI model processes the prompt sentence using its neural network layers and returns a generated text sequence.

Input: prompt sentence created in Step 11 and inference parameters.

Output: a natural-language output containing evaluations of risks, recommendations, and explanations.

Server receives the generated text and logs the interaction metadata, such as time taken and length of the response.

### Step 13:

Server parses the natural-language output into structured recommendation data.

Server applies parsing rules and natural language processing tools to identify mentions of specific intake targets, dosage ranges, cautions, and contraindications in the model output.

Server uses pattern matching for typical phrases (for example, “recommend X mg per day,” “avoid high doses of Y,” “monitor Z”) and maps them to structured fields.

Input: natural-language output from the generative AI model.

Output: structured recommendation data, including arrays of recommended intake targets with doses, lists of intake targets to avoid, and associated reasons and warnings.

Server normalizes all numeric values into internal units and ensures each structured record is associated with the correct intake target identifier.

### Step 14:

Server generates alert information and long-term health impact risk evaluation information.

Server combines the structured recommendation data with internal rule-based thresholds and domain-specific constraints. Server computes risk scores by, for example, assigning points for high dosages, known interactions between intake targets, and problematic health state evaluation results. Server then generates alert messages if risk scores exceed predefined limits. Server also produces long-term health impact risk evaluation information for each intake target by combining the output of the generative AI model with computed risk scores and historical feature trends.

Input: structured recommendation data, rule-based configuration tables, and feature records.

Output: alert information containing warnings and suggested actions, and long-term health impact risk evaluation information per intake target.

Server writes these outputs into dedicated tables and associates them with the user profile.

### Step 15:

Server prepares presentation information and sends it to the terminal.

Server assembles intake recommendation information, alert information, and long-term health impact risk evaluation information into a response object. Server ensures that each item includes a human-readable description, a recommendation status (for example, recommended, caution, avoid), and links to supporting reasons. Server serializes this response object and transmits it to the terminal over the network.

Input: recommendation records, alert records, and risk evaluation records stored in the database.

Output: a serialized presentation package sent to the terminal and a log entry indicating that the information was dispatched.

Server may apply compression or selective omission of low-importance details to optimize payload size.

### Step 16:

Terminal renders the presentation information for the user.

Terminal receives the presentation package, deserializes it, and populates user interface components. Terminal displays lists of recommended intake targets, each with suggested doses, and highlights any intake targets that should be avoided or adjusted. Terminal may color-code items according to risk level and provide expandable sections for more detailed explanations.

Input: presentation package from the server.

Output: graphical user interface views shown on the display, and internal state that reflects the current advice for the user.

Terminal may also provide buttons for the user to acknowledge warnings or to select items for purchase.

### Step 17:

User selects intake targets for purchase and confirms a transaction.

User reviews the displayed recommendations and warning messages on the terminal. User selects one or more intake targets for health support and specifies quantities and any required delivery options. User confirms the selection via an input action, such as pressing a confirmation button.

Input: on-screen options and controls rendered by the terminal.

Output: user-confirmed order details stored temporarily on the terminal, including product identifiers, quantities, and user preferences.

Terminal assembles these details into a transaction request message.

### Step 18:

Terminal sends transaction request information to the server.

Terminal transmits the transaction request message to a transaction endpoint on the server.

Terminal includes user identifiers, selected intake targets, quantities, and any supplementary data, such as selected payment method tokens.

Input: transaction request object prepared in Step 17.

Output: a network request carrying the transaction request information and an internal pending-transaction state on the terminal.

Terminal waits for the transaction result information from the server.

### Step 19:

Server executes an electronic payment process and returns transaction result information.

Server receives the transaction request information, validates product availability against a catalog, and calculates a total price. Server then communicates with an electronic payment service, sending details such as total amount, currency, and user payment token. After the payment service returns a result, server updates the purchase history information table with transaction details, including outcome and timestamps. Server constructs transaction result information that indicates success or failure and includes relevant messages.

Input: transaction request information from the terminal and responses from the electronic payment service.

Output: updated purchase history information in the database and transaction result information sent back to the terminal.

Server also logs the transaction for auditing and future analytic use.

### Step 20:

Terminal notifies the user of the transaction result and updates local state.

Terminal receives the transaction result information and displays a confirmation or error message on the screen. If the transaction is successful, terminal may show order details and estimated delivery information. If the transaction fails, terminal may prompt the user to retry or choose another payment method.

Input: transaction result information from the server.

Output: user-visible messages and updated local transaction state (for example, marking an order as completed or failed).

Terminal may store a local summary of the transaction for offline reference.

### Step 21:

Server periodically re-evaluates long-term effects and updates recommendations.

Server schedules background jobs that, at regular intervals, retrieve new biometric measurement information, usage history information, emotion state information, and purchase history information. Server recomputes feature records, generates updated explanatory text, and constructs new prompt sentences targeted at long-term effect evaluation. Server calls the generative AI model with these prompt sentences, parses the outputs, and updates long-term health impact risk evaluation information and intake recommendation information.

Input: accumulated historical time-series data and configuration for evaluation intervals.

Output: refreshed feature records, updated recommendations, and updated alert and risk evaluation records.

Server then prepares new presentation information and transmits it to the terminal, enabling the user to receive ongoing, data-driven guidance.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

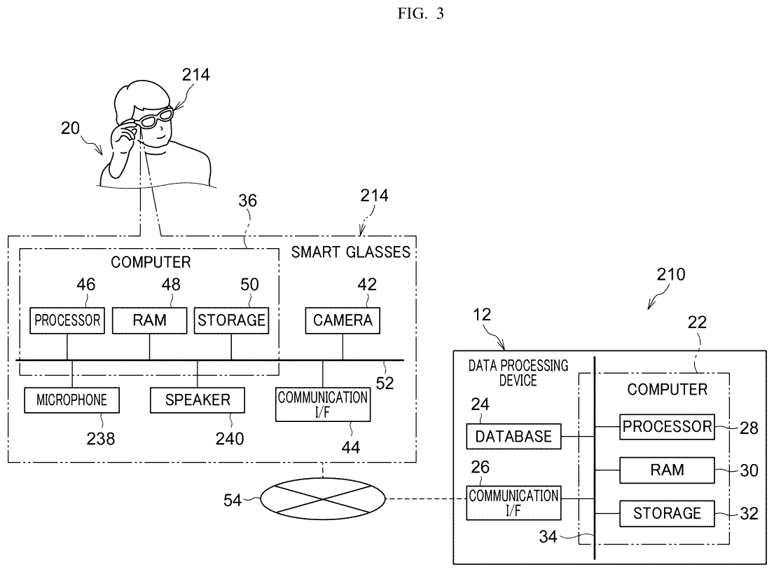

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

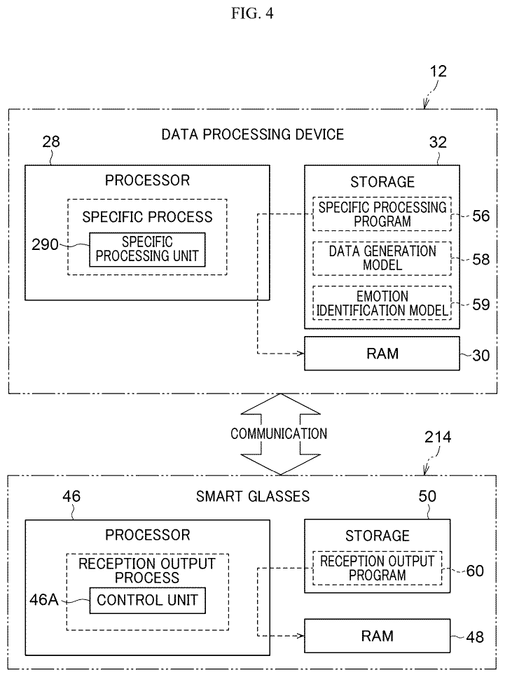

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

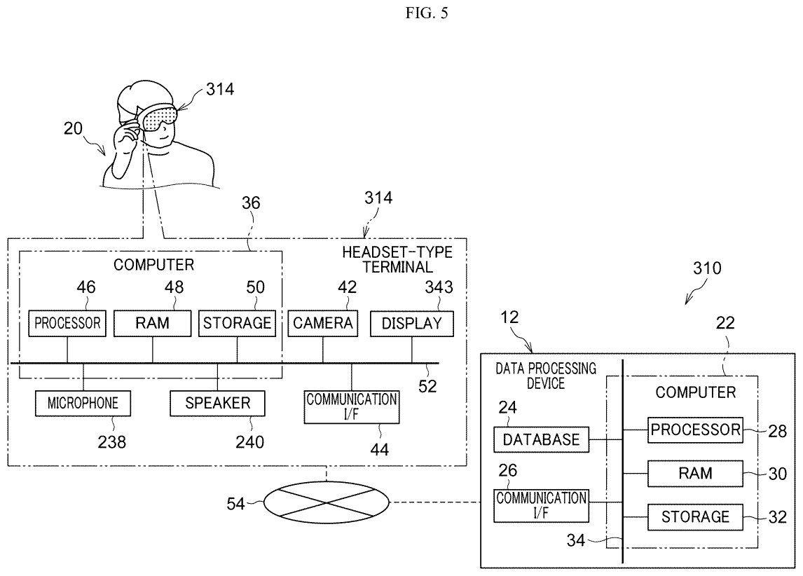

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

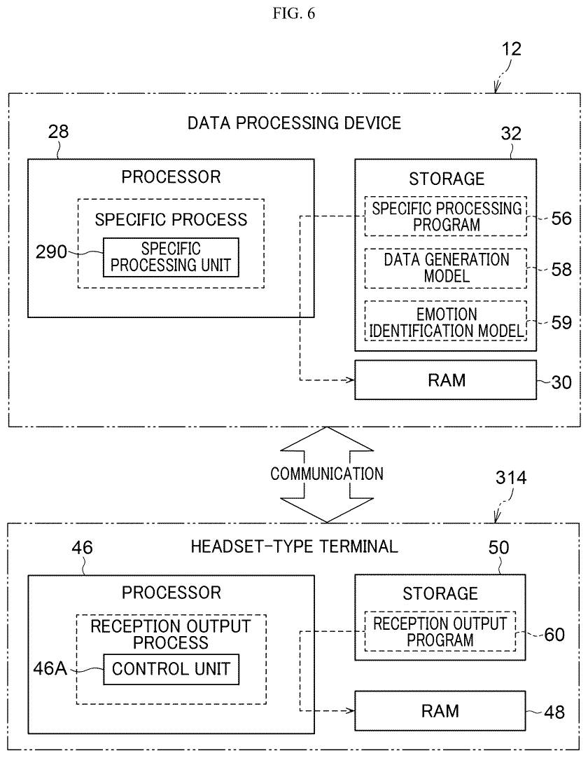

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

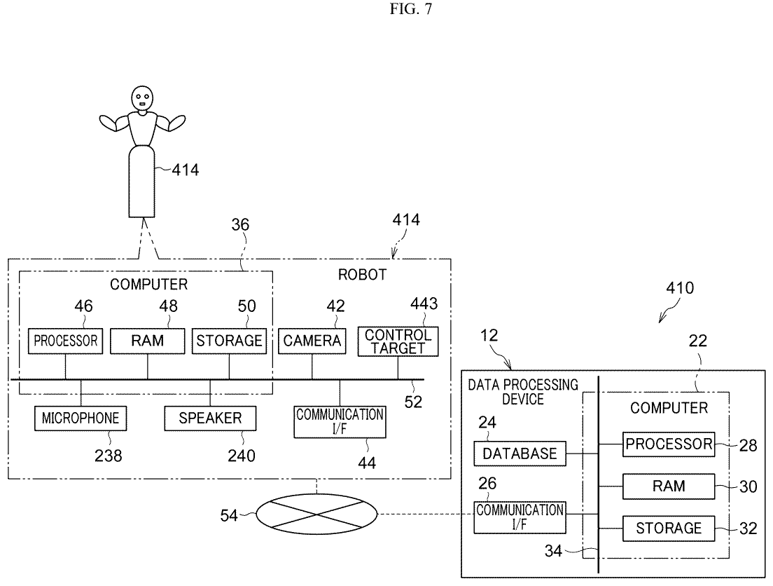

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

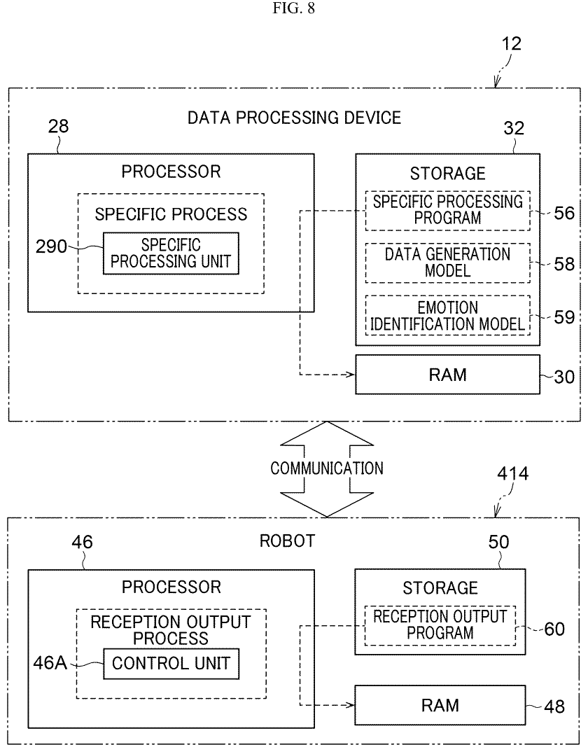

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

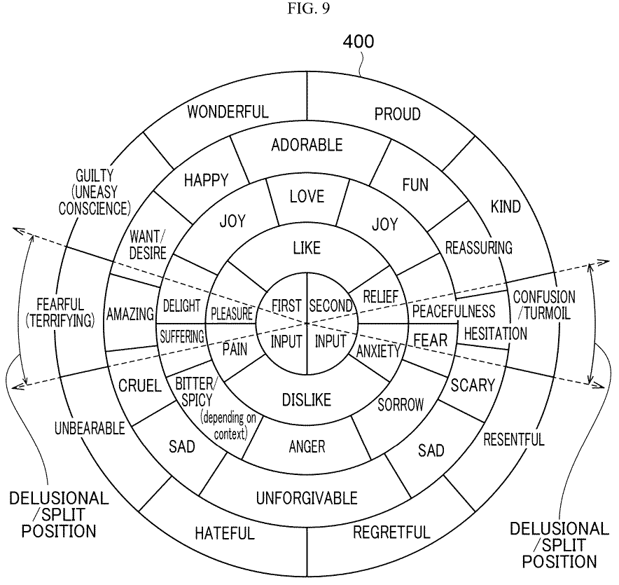

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

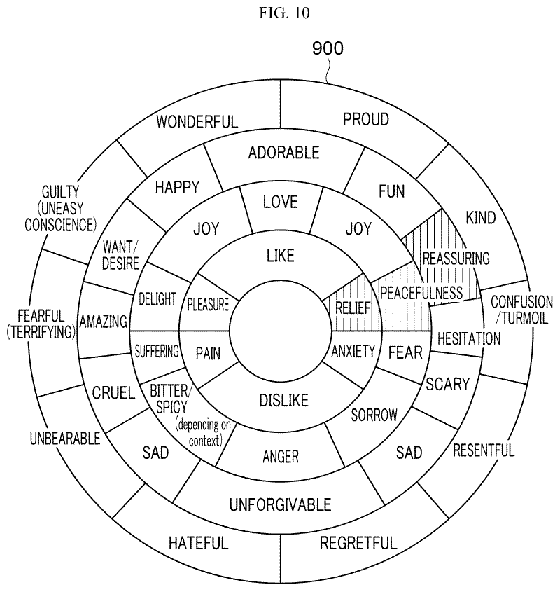

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
obtain intake information of nutrition support products from a user terminal, generate the intake information as structured information, and transmit the structured information to a server via a communication path,
receive, at the server, the structured information from the user terminal, perform format validation and content normalization on the structured information, and store normalized intake information as record information in association with individual users,
aggregate, at the server, amounts of components included in the intake information for each component over a predetermined period, and compare the aggregated amounts with medical standard information to calculate a risk level for each component and an interaction risk between components,
generate, at the server, an evaluation input sentence including the calculated result, input the evaluation input sentence as a prompt sentence into a generative AI model, and obtain, from the generative AI model, a response sentence including a natural language explanation of health risks and recommended actions,
generate, at the server, warning information including a component identifier, numerical information, a danger category, and warning content, based on the risk level and the response sentence, and transmit the warning information to the user terminal,
cause, at the user terminal, the received warning information to be presented on a display screen with a visual distinction corresponding to the danger category, update an input screen so that a user can modify the intake information in response to the presented warning information, and transmit modified intake information to the server, and
re-execute, at the server, the calculation processing and the evaluation processing using the generative AI model based on the modified intake information, and generate updated warning information.

### Supplementary 2

The system according to supplementary 1,
wherein the processor is configured to
store, at the server, as the medical standard information, intake upper limits, recommended intake amounts, and component interaction rules as storage information, and determine the risk level for each component and the interaction risk by comparing the aggregated amounts of the components with the medical standard information.

### Supplementary 3

The system according to supplementary 1,
wherein the processor is configured to
select, at the server, at least one of a template-based warning generation process and an explanation generation process using transmission of the prompt sentence to the generative AI model, in accordance with severity of the detected risk level and the interaction risk, and
generate an explanation sentence to be included in the warning information by executing the selected process.

### Application Example 1

### Supplementary 1

A system comprising a processor and a communication device,
wherein the processor is configured to
acquire, via the communication device, purchase information related to electronic transactions and intake information of nutritional support products from a user terminal device, and
register the purchase information as intake history information including component information in a storage resource, and
aggregate, for each user, an intake amount and an intake period for each nutrient component from the storage resource, generate a prompt sentence including the aggregation result and health examination information acquired from the user as input information, and input the prompt sentence into a generative AI model to cause the generative AI model to evaluate a long-term health risk, and
identify, based on evaluation information output from the generative AI model, nutritional support products that are not recommended for intake and nutritional support products that are recommended for intake, generate warning information or recommendation information including an identification result, and transmit the warning information or the recommendation information to the user terminal device via the communication device, and
recalculate an updated intake amount for each nutrient component reflecting a change in intake conditions by the user based on the storage resource and evaluation information repeatedly acquired from the generative AI model, and dynamically update input information and the prompt sentence to be input to the generative AI model to perform reevaluation.

### Supplementary 2

The system according to supplementary 1,
wherein the processor is configured to
store, in the storage resource, a daily intake amount and a cumulative intake amount for each nutrient component for each user on a time-segment basis, include structured data indicating an intake status for each time segment in the prompt sentence, and input the structured data and the prompt sentence into the generative AI model to cause the generative AI model to output a risk level and a corresponding recommended action relating to long-term excessive intake of nutrient components and interaction among a plurality of components.

### Supplementary 3

The system according to supplementary 1,
wherein the processor is configured to
perform priority assignment processing on the evaluation information output from the generative AI model according to a risk level, generate concise warning information only for nutrient components or nutritional support products whose priority is equal to or higher than a predetermined threshold, cause the concise warning information to be displayed by a notification function of the user terminal device, and store, in the storage resource, detailed evaluation content associated with the concise warning information so as to be referable.

### Example 2

### Supplementary 1

A system comprising a processor,
wherein the processor is configured to
receive, from a terminal, body information of a user and intake history information of the user,
acquire the body information and the intake history information from the terminal via an encrypted communication scheme, normalize the body information, calculate index values based on the body information, and generate feature data based on the body information and the intake history information,
generate a description text including the feature data and the intake history information, and create a prompt sentence including the description text,
input the prompt sentence into a generative artificial intelligence model and obtain, from the generative artificial intelligence model, a response text including an evaluation result of a potential health risk associated with long-term intake of nutritional supplements and including nutritional supplements recommended for intake and nutritional supplements not recommended for intake for the user,
extract, from the response text, identification information of the nutritional supplements, classification information of the nutritional supplements, recommended intake amounts, and
caution information, and generate structured data based on an extraction result,
execute rule processing on the structured data based on reference information stored in advance, correct contents of the response text of the generative artificial intelligence model,
and determine final recommended nutritional supplements for intake and final non-recommended nutritional supplements for intake, and
generate warning information and explanation information for the user based on the evaluation result of the potential health risk, and transmit the warning information and the explanation information to the terminal.

### Supplementary 2

The system according to supplementary 1,
wherein the processor is configured to
acquire continuous biometric information of the user via the terminal, combine a time-series change of the biometric information with the body information and the intake history information as time-series data, include the time-series data in the prompt sentence to be input into the generative artificial intelligence model, and update and evaluate effects of the nutritional supplements and the potential health risk.

### Supplementary 3

The system according to supplementary 1,
wherein the processor is configured to
acquire emotional state information of the user, combine the emotional state information with the body information and the intake history information, include the emotional state information in the prompt sentence to be input into the generative artificial intelligence model,
and generate an evaluation of the effects of the nutritional supplements and recommended intake contents in consideration of the emotional state information.

### Application Example 2

### Supplementary 1

A system comprising a processor,
wherein the processor is configured to
receive usage history information of a plurality of types of intake targets for health support and calculate a potential long-term health impact risk of the plurality of types of intake targets by using a generative information processing model,
generate and output alert information for a user based on a calculation result of the potential long-term health impact risk,
receive health state evaluation result information from the user and, based on the health state evaluation result information and user information including the usage history information,
extract, by using the generative information processing model, intake targets for health support that are not recommended for intake and intake targets for health support that are recommended for intake,
store the usage history information, the health state evaluation result information, biometric measurement information, emotion state information, and purchase history information in a storage device, and aggregate and preprocess the information on a time-series basis,
convert the aggregated and preprocessed information into an explanatory text expressed in natural language, configure a prompt sentence including an instruction statement that specifies an analysis target and an output format by using the explanatory text, and input the prompt sentence to the generative information processing model,
obtain a natural language output from the generative information processing model, extract, from the natural language output, names of intake targets for health support, recommended amounts, usage cautions, and contraindications, convert the extracted information into structured data, and generate, based on the structured data, intake recommendation information and long-term health impact risk evaluation information for each user,
generate presentation information for supporting selection and dosage adjustment of the intake targets for health support by the user based on the intake recommendation information and the long-term health impact risk evaluation information, and transmit the presentation information to a terminal device via a network,
receive transaction request information from the terminal device, execute an electronic payment process based on product information of the intake targets for health support included in the intake recommendation information and the transaction request information, and return transaction result information corresponding to a payment result to the terminal device, and
analyze histories of the usage history information, the biometric measurement information,
and the emotion state information over a predetermined period, automatically generate, for the generative information processing model, a prompt sentence for periodically re-evaluating long-term effects and long-term risks for each intake target for health support, and output a re-evaluation result as follow-up feedback information for the user.

### Supplementary 2

The system according to supplementary 1,
wherein the processor is configured to
calculate statistical values of biometric indicators and occurrence frequencies of emotion states before and after a start of use of each intake target for health support based on the aggregated and preprocessed usage history information, biometric measurement information,
and emotion state information, and include the statistical values and the occurrence frequencies in the prompt sentence so as to cause the generative information processing model to evaluate long-term effects of the intake targets for health support.

### Supplementary 3

The system according to supplementary 1,
wherein the processor is configured to
dynamically change the prompt sentence for the generative information processing model in accordance with at least one generation purpose selected from alert information, intake recommendation information, and evaluation results of long-term effects, and generate and transmit a plurality of types of the prompt sentences for the same usage history information and the same health state evaluation result information, thereby obtaining complementary analysis results from the generative information processing model.

What is claimed is:

1. A system comprising:
circuitry configured to:
receive, via a packet-switched network, intake record data from a terminal device, perform format validation and content normalization on the intake record data, and store normalized intake record data in a storage device in association with a user identifier;
aggregate, for each component identifier in the normalized intake record data, numerical intake values over a predetermined temporal period to produce aggregated component data;
compare the aggregated component data with stored reference parameter data to compute a risk level value for each component identifier and an interaction risk value for combinations of component identifiers;
construct a structured prompt for a generative neural network model by formatting the aggregated component data, the risk level values, and the interaction risk values into an evaluation input sentence, and transmit the structured prompt to an information processing apparatus hosting the generative neural network model via the packet-switched network;
receive a generated output token sequence from the generative neural network model, the generated output token sequence comprising a natural language explanation of identified risks and action data;
generate warning data comprising the component identifiers, the risk level values, a danger category for each component identifier, and warning content derived from the generated output token sequence, and transmit the warning data to the terminal device via the packet-switched network to cause rendering of the warning data on a display of the terminal device; and
receive modified intake record data from the terminal device, re-execute the aggregation, comparison, structured prompt construction, and transmission to the generative neural network model based on the modified intake record data, and generate updated warning data.

2. The system according to claim 1, wherein the stored reference parameter data comprises intake upper limit values, recommended intake amount values, and component interaction rules stored in the storage device, and the circuitry determines the risk level value by computing a ratio between each aggregated component value and a corresponding intake upper limit value.

3. The system according to claim 2, wherein the circuitry evaluates the component interaction rules by applying Boolean logic on presence flags and intake threshold values for combinations of component identifiers to determine the interaction risk value.

4. The system according to claim 3, wherein the circuitry selects between a template-based warning generation path and a generative-neural-network-based explanation path based on severity of the risk level values and the interaction risk values, the template-based path inserting numeric values into predefined text templates when all risk level values are below a configured severity threshold.

5. The system according to claim 4, wherein the generative neural network model processes the structured prompt through multiple transformer layers, computing attention scores and updating hidden states to predict subsequent tokens according to a language modeling objective, and the circuitry decodes the predicted tokens into the generated output token sequence.

6. The system according to claim 5, wherein the intake record data comprises intake information of a plurality of nutritional supplements, and the stored reference parameter data comprises medically established tolerable upper intake limits and recommended daily allowances for nutrient components of the nutritional supplements.

7. The system according to claim 6, wherein the warning data further comprises an indication of nutritional supplements that are not recommended for intake and nutritional supplements that are recommended for intake, derived from the risk level values and the generated output token sequence.

8. The system according to claim 7, wherein the circuitry is further configured to:
receive health examination result data from the terminal device comprising at least blood test values and physical measurement values, and incorporate the health examination result data into the structured prompt to cause the generative neural network model to generate individualized risk evaluation and recommendation data based on the health examination result data.

9. The system according to claim 1, wherein the circuitry is further configured to:
receive time-series biometric measurement data from the terminal device, the time-series biometric measurement data comprising periodic physiological measurements acquired from a sensing device, compute summary statistics over temporal windows from the time-series biometric measurement data, and incorporate the summary statistics into the structured prompt to cause the generative neural network model to evaluate an effect of the intake record data by correlating the summary statistics with the aggregated component data.

10. The system according to claim 9, wherein the time-series biometric measurement data comprises at least heart rate values and blood pressure values, and the summary statistics comprise average values, variability measures, and trend coefficients computed over the temporal windows.

11. The system according to claim 10, wherein the circuitry aligns the time-series biometric measurement data to a common time grid by resampling to fixed intervals, interpolating missing values, and joining the time-series biometric measurement data with intake event timestamps using window functions to associate pre-intake and post-intake biometric measurements with corresponding intake events.

12. The system according to claim 1, wherein the circuitry is further configured to:
receive emotional state data from the terminal device, the emotional state data comprising a categorical emotion label and an associated probability value, and incorporate the emotional state data into the structured prompt to cause the generative neural network model to evaluate an effect of the intake record data by combining the emotional state data with the aggregated component data.

13. The system according to claim 12, wherein the circuitry computes occurrence frequencies of emotion categories within pre-intake and post-intake temporal windows for each component identifier, and includes the occurrence frequencies in the structured prompt.

14. The system according to claim 1, wherein the circuitry is further configured to:
at regular intervals, retrieve updated intake record data, time-series biometric measurement data, and emotional state data from the storage device, recompute the aggregated component data and the summary statistics, construct updated structured prompts targeted at long-term effect evaluation, and transmit the updated structured prompts to the generative neural network model to obtain updated risk evaluation data.

15. The system according to claim 1, wherein the warning data is rendered on the display of the terminal device with a visual distinction corresponding to the danger category, and the terminal device updates an input interface to enable the user to modify the intake record data in response to the rendered warning data.

16. The system according to claim 1, wherein the content normalization comprises unifying measurement units for each component identifier using predefined conversion rules, aligning free-text component names to canonical component identifiers using string normalization and table lookup operations, and clamping values outside a predetermined physiological range.

17. The system according to claim 1, wherein the circuitry is further configured to:
store log data associating the structured prompt, the generated output token sequence, the warning data, and the modified intake record data, and update wording of a prompt template or retrieval conditions for the stored reference parameter data based on analysis of the log data.

18. A system comprising:
circuitry configured to:
receive, via a packet-switched network, intake record data comprising intake information of a plurality of nutritional supplements from a terminal device, normalize the intake record data, and store normalized intake record data in a storage device;
aggregate numerical intake values per nutrient component over a predetermined temporal period and compare aggregated values with medically established reference parameters to compute risk level values and interaction risk values;
receive health examination result data comprising blood test values and physical measurement values from the terminal device;
construct a structured prompt for a generative neural network model incorporating the aggregated values, the risk level values, the interaction risk values, and the health examination result data, and transmit the structured prompt to an information processing apparatus hosting the generative neural network model via the packet-switched network;
receive a generated output token sequence and generate warning data comprising nutritional supplements not recommended for intake, nutritional supplements recommended for intake, and a natural language risk explanation;
receive time-series biometric measurement data and emotional state data from the terminal device, compute summary statistics and occurrence frequencies, and incorporate the summary statistics and occurrence frequencies into subsequent structured prompts to cause the generative neural network model to evaluate effects of the nutritional supplements; and
transmit the warning data to the terminal device via the packet-switched network to cause rendering on a display of the terminal device, and re-execute evaluation processing when modified intake record data is received.

19. The system according to claim 18, wherein the circuitry periodically retrieves updated intake record data, biometric measurement data, and emotional state data, recomputes aggregated values and summary statistics, constructs updated structured prompts for long-term effect evaluation, and transmits the updated structured prompts to the generative neural network model.

20. A method performed by circuitry, the method comprising:
receiving, via a packet-switched network, intake record data from a terminal device, performing format validation and content normalization on the intake record data, and storing normalized intake record data in a storage device;
aggregating, for each component identifier, numerical intake values over a predetermined temporal period to produce aggregated component data;
comparing the aggregated component data with stored reference parameter data to compute a risk level value for each component identifier and an interaction risk value for combinations of component identifiers;
constructing a structured prompt for a generative neural network model by formatting the aggregated component data, the risk level values, and the interaction risk values into an evaluation input sentence, and transmitting the structured prompt to an information processing apparatus hosting the generative neural network model via the packet-switched network;
receiving a generated output token sequence comprising a natural language explanation of identified risks and action data;
generating warning data comprising the component identifiers, the risk level values, a danger category, and warning content derived from the generated output token sequence, and transmitting the warning data to the terminal device via the packet-switched network to cause rendering on a display of the terminal device; and
receiving modified intake record data from the terminal device and re-executing the aggregating, comparing, constructing, and transmitting based on the modified intake record data to generate updated warning data.
